(19) United States
(12) Patent Application Publication
(10) Pub. No.: US 2011/0212028 A1
(43) Pub. Date: Sep. 1, 2011

(54) CHEMICALLY MODIFIED CELL-PENETRATING PEPTIDES FOR IMPROVED DELIVERY OF GENE MODULATING COMPOUNDS

(76) Inventors: Kariem Ahmed, Stockholm (SE); Samir El Andaloussi, Stockholm (SE); Peter Gutrestam, Stockholm (SE); Mattias Hallbrink, Stockholm (SE); Henrik Johansson, Solna (SE); Ulo Langel, Stockholm (SE); Taavi Lehto, Tartu (EE); Maria Lindgren, Stockholm (SE); Imre Mager, Jarvamaa (EE); Rannar Sillard, Enebyberg (SE); Katri Rosenthal Aizman, Alvsjo (SE); Ulf Tedebark, Jarfalla (SE); Per Lundin, Johanneshov (SE)

(21) Appl. No.: 13/119,398

(22) PCT Filed: Sep. 16, 2009
(86) PCT No.: PCT/SE2009/051032

§ 371 Date: May 18, 2011

Related U.S. Application Data
(60) Provisional application No. 61/136,574, filed on Sep. 16, 2008.

(51) Int. Cl. A61K 49/00; C07K 2/00; C07K 7/08; C07K 7/06; C07K 14/00; C07K 9/00; C12N 15/63; A61K 47/42; C12N 5/00; A61P 35/00; A61P 31/04

(52) U.S. Cl. 424/9.1; 530/300; 530/326; 530/327; 530/328; 530/324; 530/322; 435/320.1; 514/773; 435/375

(57) ABSTRACT

The present invention relates to a system for intracellular delivery of a cargo comprising at least one component A chosen from aliphatic linear or branched moieties with at least 4 carbon atoms and/or cyclic ring systems comprising 2-4 rings which may contain several hetero atoms chosen from N, S, O and P, wherein component(s) A is (are) attached to a cell penetrating peptide B and/or a non-peptide analogue thereof. It also relates to methods of using the system in diagnosis of diseases, as research tool and as a targeting system, a composition comprising the system and especially a pharmaceutical composition a material covered with the system and a material having the delivery systems into the material. Finally it relates to novel peptides.

### FIELD OF THE INVENTION

The present invention relates to a system for intracellular delivery of a cargo comprising at least one component A chosen from aliphatic linear or branched moieties with at least 4 carbon and/or cyclic ring systems comprising 2-4 rings which may contain several hetero atoms chosen from N, S, O and P, wherein component(s) A is (are) attached to a cell penetrating peptide B and/or a non-peptide analogue thereof.

It also relates to the use of the system in diagnosis of deceases, as research tool and as a targeting system, a composition comprising the system and especially a pharmaceutical composition a material covered with the system and a material having the delivery systems into the material. Finally it relates to novel peptides.

### BACKGROUND OF THE INVENTION

The hydrophobic plasma membrane constitutes an essential barrier for cells in living animals, allowing the constitutive and regulated influx of essential molecules while preventing access to the interior of cells of other macromolecules. Although being pivotal for the maintenance of cells, the inability to cross the plasma membrane is still one of the major obstacles to overcome in order to progress current drug development.

During the past 40 years, several oligonucleotide (ON)-based methods have been developed with the purpose of manipulating gene expression. The basic method involves the use of bacterial plasmids for expression of genes of interest. In addition, to evaluate functional aspects of different genes, this is a highly appealing strategy to utilize in clinical settings, i.e. gene therapy. Gene therapy was originally thought to serve as corrective treatment for inherited genetic diseases. However, over the past 15 years, experimental gene therapy for cancer has become a frequent application although other acquired diseases have also been investigated [1].

Other versatile approaches utilizing shorter ON-sequences to interfere with gene expression have emerged. Antisense approaches based on short interfering RNAs (siRNAs) that are utilized to confer gene silencing and splice correcting ONs (SCOs), applied for the manipulation of splicing patterns, have recently been rigorously exploited [2,3]. Although being efficient compounds for regulating gene expression, their hydrophilic nature prohibits cellular internalization.

Despite the great potential gene therapy holds for future treatment of various disorders, it suffers from some severe drawbacks. First, plasmids are large, usually exceeding one MDa in size, making them impermeable over cellular membranes. Secondly, viruses have been used to confer cellular internalization of therapeutic genes in clinical trials. Albeit providing an effective means of delivering genes, they might cause severe immunological responses. Thus, in order to progress current gene therapy, safer delivery systems are required, preferably not reliant on the use of viruses.

The search for efficient non-viral delivery vectors has therefore intensified. In the field today, the vectors based on cationic liposomes or polycationic polymers have been employed and these are highly efficient for transfection of commonly used cell lines. However, a great number of these vectors are either sensitive to serum proteins, are unable to transfect the entire cell populations, are inefficient in “hard to transfect” cells, or are simply too toxic. For the vectors on the market today it seems to be a direct correlation between high efficacy and high cytotoxicity. Therefore, there is an urgent need to find delivery vehicles that can overcome the above mentioned problems.

Cell-penetrating peptides (CPPs) are a class of peptides that has gained increasing focus the last years. This ensues as a result of their remarkable ability to convey various, otherwise impermeable, macromolecules across the plasma membrane of cells in a relatively non-toxic fashion, as reviewed in [4]. The peptides are usually less than 30 amino acids (aa) in length with a cationic and/or amphipathic nature and have been extensively applied for delivery of various ONs both in vitro and in vivo [5]. Even though the peptides are non-toxic in general, there are some problems associated with their use [6]. One shortcoming with the CPP technology, in terms of ON-delivery, is that peptides usually need a covalent attachment to ONs, which is a cumbersome procedure and high concentrations of peptide conjugates are generally needed to obtain a significant biological response [7,8]. A few studies have convincingly shown that a non-covalent co-incubation strategy of simply mixing CPPs with ONs works efficiently and in a non-toxic fashion. When using the co-incubation strategy with unmodified CPPs, it seems that the complexes remain trapped inside endosomes and are therefore unable to exert a biological response [9]. Ideally, CPPs would be designed to more efficiently escape endosomal compartments following endocytosis thereby allowing them to be non-covalently complexed with oligonucleotides or plasmids. Attempts have been made to combine the use of CPPs with known transfection reagents to reduce the amount of transfection regent needed to obtain biological responses or CPPs have been co-added with known fusogenic peptides. Another strategy has been to co-add the lysosomotrophic agent chloroquine at high concentrations to increase the efficacy of the CPP/ON complexes, which significantly increases transfections but is limited to in vitro use and furthermore, the high concentrations of chloroquine needed raises toxicity concerns.

A related patent, US 2007/0059353 discloses a liposome having cellular and nuclear entry ability. The provided liposome has on its surface a peptide comprising multiple consecutive arginine residues, and specifically a liposome is provided wherein the peptide is modified with a hydrophobic group or hydrophobic compound and the hydrophobic group or hydrophobic compound is inserted into a lipid bilayer so that the peptide is exposed on the surface of the bilayer. The problem with this delivery system, apart from the difficulty of constructing such complex vectors, is that they are based on liposomes. Several groups have reported on alterations in gene expression profiles after transfections with liposome-based delivery systems which greatly hamper their use. In addition, oligoarginines are prone to remain bound to endosomal compartments and are therefore not optimal for delivery. An improved strategy would be to chemically modify newly designed or existing CPPs with one or more chemical entities that could promote endosomal escape.

The drug of choice today for endosomal escape is Chloroquine (CQ) and its analogues. It is a, as it is also called, lysosomotropic agent, inhibiting endosome acidification, leading, at higher concentrations, to endosomal swelling and rupture.

There are several U.S. patents disclosing chloroquine for use against a variety of diseases either alone or in combination with other drugs. For instance, U.S. Pat. No. 4,181,725 and A. M. Krieg, et al, U.S. Patent Applic. 20040009949 disclose the use of chloroquine for treating various autoimmune diseases in combination with inhibitory nucleic acids.

The ability of chloroquine to act as “lysosomotropic”agent to enable release of substances from cellular endosomes/lysosomes is well-documented. [Marches, 2004; A. Cuatraro 1990 etc]. Nevertheless, in vivo use of chloroquine was claimed to be prohibited by its toxicity, as high concentration of free chloroquine needs to be administrated to reach endosomes. (Citing J. M. Benns, et al, 1.sup.st paragraph, Bioconj. Chem. 11, 637-645, (2000): “Although chloroquine has proven to aid in the release of the plasmid DNA into the cytoplasm, it has been found to be toxic and thus cannot be used in vivo.”)

Recent USPTO Application #: 20070166281 entitled “Chloroquine coupled antibodies and other proteins with methods for their synthesis” discloses coupling of chloroquine and thereof derived structures to different carrier compositions that contain biocleavable linkages allowing release of chloroquines under controlled conditions. Application #: 20070166281 is aimed to provide controlled release of the chloroquines from protein or peptide active agent or antibody after the carrier has reached its site of action.

US2006/0040879 Kosak and colleagues patent discloses compositions and methods for preparing chloroquine-coupled nucleic acid compositions. The prior art has shown that chloroquine given as free drug in high enough concentration, enhances the release of various agents from cellular endosomes into the cytoplasm. The purpose of these compositions is to provide a controlled amount of chloroquine at the same site where the nucleic acid needs to be released, thereby reducing the overall dosage needed. This patent is aimed at achieving controlled release of chloroquine conjugated to nucleic acid compositions, this is not the subject of the present invention, but rather to enhance and simplify delivery in gene therapy in vitro and in vivo.

### SUMMARY OF THE INVENTION

The present invention provides a system for intracellular cargo delivery comprising a new series of molecules that overcome the described drawbacks for non-covalent gene-delivery, ie low and heterogeneous delivery as well as toxicity. The present invention is in no need of biocleavable linkers to cleave chloroquine analogues. The system according to the present invention comprises irreversibly chloroquine-coupled compounds.

The system comprises compounds which are improved CPPs with fatty acid modifications which can be utilized for efficient delivery of a wide variety of ONs, without the toxicity of the delivery agents on the market today. The next generation of further derivatised CPPs can both efficiently deliver the drug load into all of the cells in a population as well as releasing the ONs from their entrapment in endosomes. The claims of the invention describe the modified and derivatised delivery peptides and their tested applications; enhanced transfection, splice correction as well as siRNA delivery.

### BRIEF DESCRIPTION OF THE DRAWINGS

FIG. 1 Pre-mRNA of the modified luciferase gene. The intron 2 from the β-globin gene carrying a point mutation at nucleotide 705 is inserted into the luciferase gene. Blockage of this site with SCO redirects splicing towards the functional mRNA. Thus, this generates a positive biological read-out that relies on the fact that SCOs reaches the nucleus of cells.

FIG. 2 Quantitative uptake and splice correction after treatment with unmodified CPPs or Lipofectamine 2000 complexed with 200 nM SCO. A) Uptake after 1 h of Cy5-SCO complexed with different CPPs at molar ratios of 1:5, 1:10, and 1:20. B) Splice correction after treatment with same set of complexes as in A. Treatments were carried out for 2 h in serum free DMEM after which media was replaced for full growth media for additionally 20 h. C) Splice correction after treatment with increasing concentration of SCO, using Lipofectamine 2000 according to manufacturers protocol. D) Same as in B but with co-treatment of 75 μM chloroquine (CQ) that prohibits acidification of endosomes and, thus, promotes endosomal escape. The results clearly show that the unmodified CPPs confer uptake of SCOs but are unable to induce splice correction. Since chloroquine significantly increases splice correction it is plausible to assume that CPP/SCO complexes exclusively reside in endosomal compartments.

FIG. 3 Gel retardation assay. Complexes were formed as previously mentioned and run on a PAGE gel for 1 h. Peptides were mixed with phosphorothioate 2°O-methyl RNA (i.e SCO) at molar ratio 5, 10, and 20. Material that are retained in wells are defined as peptide/SCO complexes. M918, tightly followed by TP10, are the two most potent peptides in forming complexes.

FIG. 4 Uptake and splice correction using stearyl modified CPPs in complex with SCOs. A) Quantitative uptake was conducted as in FIG. 2a. Uptake of Cy5-SCO complexed with stearylated Arg9 or pentratin was overall higher than for the PepFect peptides. B) Same peptides and ratios were used for splice correction. Interestingly, PepFect 3 induced a dramatic increase in correct splicing while stearylated penetratin and Arg9 had negligible activity. PepFect 2 had some activity despite the low observed uptake.

FIG. 5 a) General structure of the Pepfect delivery system where A=alifatic fatty acid or a di-tetra ringsystem, 1-8 copies, some possibly on a branched spacer, B=cell-penetrating peptide or non-peptide analogue thereof and C=targeting moiety such as homing peptide or aptamer, can be by non-covalent attachment. b) example of fatty acid: schematic drawing of stearic acid c) example of ringsystem: N-(2-aminoethyl)-N-methyl-N′-[7-(trifluoromethyl)-quinolin-4-yl]ethane-1,2-diamine. D) schematic structure and names of the Pepfect delivery systems.

FIG. 6 A comparison between TP10, TP10 together with chloroquine (CQ), and PepFect 3 (stearylated version of TP10) for their ability to promote SCO mediated splice correction. At low molar ratio, 1:5, PepFect 3 induces almost a 40-fold increase in correctly spliced luciferase, which could be compared to a 2-fold increase for TP10. However, there seem to be space for improvement of PepFect 3, as chloroquine co-treatment with TP10 generates a 70-fold increase in splicing.

FIG. 7 Comparison between PepFect3 (N-terminally stearylated) and PepFect4 (orthogonally stearylated on Lys⁷) for the delivery of SCOs. Both peptides promote a dose-dependent increase in SCO-mediated splice correction with PepFect4 being most potent. Cells were treated for 4 h in serum free media after which cells were replaced with full growth media for additionally 20 h. After cell lysis and measurements of luminescence data were normalized to protein content in each well and presented as fold-increase in splicing over untreated cells. PepFect3 was complexed at molar ratio 10 while PepFect4 was complexed at molar ratio 7 over SCO.

FIG. 8 Comparison of PepFect3 and 4 with Lipofectamine 2000 using 200 nM SCO. While PepFect 3 is slightly less active than the commercial liposome based transfection reagent Lipofectamine 2000, PepFect4 exceeds that activity. If comparing with the pre-clinically used CPP-morpholino conjugate, (RXR)4-PMO, used at 5 μM concentration, both PepFect peptides appear superior at 25-times lower SCO concentrations. Experiments were performed as in FIG. 6.

FIG. 9 Transfection of an luciferase encoding plasmid using PepFect peptides in HeLa cells. All transfection complexes were formed according to the materials and methods section and gene expression was monitored 24 h after treatment. A, C, and E show the luciferase expression from pGL3 plasmid complexed with PepFect 1, 2, or 3 respectively at different molar ratios. D) Comparison between the peptides at a charge ratio of 1:1. B) Lucifease expression after transfection with the positive control Lipofectamine 2000.

FIG. 10 Transfection of luciferase-encoding plasmids using PepFect3 compared to Lipofectamine 2000 in CHO cells after 24 h incubation. Complexes of PF3 and plasmid were prepared at different charge ratios ranging from CR 0.5-2 and compared to the transfection efficiency of Lipofectamine 2000 applied according to manufacturers protocol. Results are presented as fold increase in gene expression compared to cells treated with plasmid only. The graph clearly illustrates that at CRs above 1, PF3 is more active than Lipofectamine 2000.

FIG. 11 Transfection of EGFP-expressing plasmid using PF3 or Lipofectamine measured after 24 h in CHO cells. PF3 was complexed with plasmid at different CRs and compared with Lipofectamine 2000. The results suggest that although overall transfections with Lipofectamine 2000 is in parity with PF3, the number of cells transfected are significantly higher when exploiting the PF3 peptide. These experiments were performed in serum free media.

FIG. 12 Plasmid transfection in HEK293 cells at different cell confluencies. Experiment was performed essentially as in FIG. 10, using PF4 compared to Lipofectamine 2000. Strikingly, transfection is increased at higher cell densities and at CRs above 1, PF4 is significantly more active than Lipofectamine 2000 in terms on conveying plasmids into kidney cells.

FIG. 13 Metabolic activity in HeLa cells treated for 24 h either with PF3 at different CRs or Lipofectamine. Y-axis is % of metabolicly active cells relative to untreated cells. The results from the WST-1 assay, which measures the metabolic activity in mitochondria, it is clear that whereas PF3 complexed with plasmids has negligible effects on proliferation, significant long-term toxicities are observed after treatment with Lipofectamine 2000 according to manufacturers protocol. Complexes were prepared as in the gene delivery assays but treatments were performed in 96-well format rather than in 24-well plates.

FIG. 14 Dose-response curve comparing the efficacy of PepFect5 with Lipofectamine 2000 for the delivery of anti-miR21 ONs. Both reagents appear to be equally efficient when using PepFect5 at a very low molar ratio of 2 over the ON

FIG. 15 A comparison of PepFect5 and Lipofectamine 2000 for the delivery of anti-miR21 ON at 100 nM concentration. When using PepFect5 at a molar ratio (MR) of 5, the peptide is significantly more efficient than Lipofectamine 2000. As expected, un-vectorized ON has no activity.

FIG. 16 A dose-response curve on luciferse down-regulation in luciferase-stable HeLa cells using either Lipofectamine 2000 or PepFect5 as delivery agent for siRNA. PepFect5 was complexed with siRNA at a molar ratio of 40 and the gene silencing observed using 25 nM siRNA was in parity with that of Lipofectamine 2000 using 100 nM siRNA.

FIG. 17 Comparison of the efficacy of PepFect 5 (PF5) and PF6, complexed at two different molar ratios, to convey siRNA targeting luciferase in luciferase stable BHK21 cells. PF6 is superior to PF5, especially at low siRNA concentration despite the lower amount of peptide used with PF6.

FIG. 18 Dose-response curve of PF6/siRNA complexes formed at high molar ratio (80) on luciferase down-regulation in luciferase stable osteosarcoma cells (U2OS). Siginificant RNAi is observed at as low concentration as 5 nM.

FIG. 19 A dose-response curve on PF6/siRNA complex transfections performed in full growth media. A dose-dependent decrease in luciferase expression in luciferase stable BHK21 cells was observed with increasing siRNA concentrations. Here, complexes were preformed and simply added to the growth media. Luciferase expression was assessed 24 h after transfection.

FIG. 20 RNAi in BHK21 cells after 24 h treatment with PF6 complexed with 50 nM siRNA at different molar ratios. Even at such low MR as 10, it is possible to obtain more than 80% down-regulation of luciferase in luciferase-stable BHK21 cells. This is a stronger RNAi effect than what is typically possible to obtain with Lipofectamine 2000 or any other Liposome-based delivery system. Interestingly, between MR20 and MR40, the difference in response is rather low. This makes the delivery system more user-friendly compared to Lipofectamine 2000, where small changes in amounts taken for transfections have drastic impact on the transfection efficiency.

FIG. 21 Down-regulation in EGFP stable CHO cells after treatment with siRNA targeting EGFP complexed with Lipofectamine 2000 or PepFect6 measured by FACS. a) represents the fluorescence from untreated cells (blue). b) panel shows EGFP expression after treatment with 100 nM siRNA and Lipofectamine 2000. The blue population represents non down-regulated cells and the red population represents EGFP down-regulated cells. The lower left panel c) is cells treated with only 25 nM siRNA complexed with PF6 (molar ratio 40). As seen, 90-95% of cells have lower expression of EGFP (red). Finally, the lower right panel d) show EGFP expression after treatment with 100 nM siRNA complexed with PF6 in full serum media. 48 h after treatment, approximately 98% of cells have negligible EGFP expression. In conclusion, PF6 is far more potent than Lipofectamine 2000 in terms of inducing RNAi, and it seems that the delivery is ubiquitous sine almost complete RNAi is observed. Even in full serum media, the effect of the peptide is more significant than Lipofectamine 2000.

FIG. 22 Confocal microscopy analysis in living EGFP-CHO cells treated with siRNA for 48 h. 100 nM siRNA was used as a negative control and Lipofectamine 2000 complexed with 100 nM siRNA was used as positive control. Naked siRNA has very little effect on the EGFP expression whereas Lipofectamine 2000, as expected, induce RNAi in some cells. In line with the FACS histograms presented in FIG. 21, PF6 co-incubated with 50 nM siRNA induces complete RNAi both in serum free media and in complete growth media. Again, it seems that the PF6/siRNA particles enters cells in a ubiquitous manner compared to Lipofectamine 2000 that only enters a certain fraction of cells, most likely the dividing cells.

FIG. 23 Flow cytometry analysis of RNAi decay kinetics following a single siRNA treatment in EGFP-CHO cells. PF6/siRNA particles were formulated at the given concentrations of siRNA and treatment where performed in serum (FM) or serum free media (SFM). The effect was then compared to the RNAi induced by 100 nM siRNA complexed with Lipofectamine 2000 or Oligofectamine. The results clearly show that independent on siRNA concentration, PF6 induces almost complete RNAi already after 24 h. This should be compared to a 20% and 55% knock-down observed with Oligofectamine and Lipofectamine 2000, respectively. Furthermore, at optimal conditions, the RNAi response persists for 4-5 days when using PF6. MR, molar ratio.

FIG. 24 Down-regulation of HPRT1 mRNA in osteosarcoma cells. Significant knock-down is observed at both molar ratios of PF6 at all concentrations while Lipofectamine 2000 fail to induce any RNAi at 50 μM siRNA concentrations. In these experiments, Dicer-substrate siRNAs (longer versions that are processed by dicer) were used. Cells were treated in serum free media and mRNA levels were analyzed by RT-PCR 24 h post transfection.

FIG. 25 HPRT1 knock-down in human SHSY5Y neuroblastoma cells following 20 h of siRNA treatment. PF6/siRNA particles where formulated at two different molar ratios and serial diluted to give a final treatment concentration of 100, 50 and 25 nM siRNA. Treatments were performed in full growth media and the RNAi response was compared to that induced by 100 nM siRNA complexed with Lipofectamine 2000. At MR40 of PF6 over siRNA, the RNAi response is significantly stronger even at 25 nM siRNA concentration as compared to Lipofectamine 2000 using 100 nM siRNA. This shows that the PF-system is not only active in serum but also that it efficiently transfect rather “hard to transfect” cells in a ubiquitous manner.

FIG. 26 RNAi in rat primary mixed glial cell culture following treatment with siRNA targeting HPRT1. Both in serum and under serum free conditions, PF6 is significantly more potent than Lipofectamine 2000 to confer siRNA-mediated gene silencing.

FIG. 27 Transfection of luciferase expressing plasmid in CHO cells after 24 h using either Lipofectamine 2000 or PF6. At a charge ratio (CR) of 2 of peptide over plasmid, one log higher transfections are observed with PF6 compared to Lipofectamine in serum free media. Even in serum media, at CR4, the peptide promotes plasmid transfections twice as efficiently as Lipofectamine 2000.

FIG. 28 Transfection of EGFP plasmids in Jurkat suspension cells. Whereas Lipofectamine 2000 is almost completely inactive, significant transfections were observed using PF6, especially at higher charge ratios. EGFP expression was assessed 24 h after transfection in serum free media by FACS. b) Histogram of corresponding Jurkat transfection. Although the overall transfection levels are rather low, PF6 has the ability to transfect a large fraction of cells in the population. c) Jurkat plasmid transfection not dependent on cell confluenncy. Intriguingly, with PF6 it is possible to transfect the majority of cells independent of confluency.

FIG. 29 Splice correction in HeLa cells after treatment with an PF5 analog complexed with SCOs. In this case, TP10 with lysine branching orthogonally with four 1-naftoxy acetic acid was used instead of the fluoroquine moiety in order to assess the importance of the fusogenic properties. This figure shows the fold increase in luciferase expression relative to control in Hela pluc 705 cells treated at different peptide/SCO molar ratios ranging from 5 to 25 in both serum and serum free media, using 200 nM SCO. The results suggest that this peptide is significantly less active, reaching 12-fold increases in splicing which could be compared to the 100-fold increase observed for PF5 previously.

FIG. 30 A, Splice correction in HeLa cells 24 h after treatment with Pepfect 14/SCO complexes. PF14 complexes are highly active independent of molar ratio and prescence of serum. At 200 nM SCO concentration, PF14 is significantly more active in terms of conveying SCOs inside cells compared to Lipofectamine 2000. Even at such low concentration as 50 nM SCO, a 50-fold increase in splicing was observed. B) This figure shows the percent of luciferase expression relative to control after treatment of BHK-21 cells stably expresssing the luciferase gene with different petide/siRNA molar ratios ranging from 30 to 40 in serum media, and compared to lipofectamine trasfection according to the manufacturer protocol. At a molar ratio of 35, PF14 is in fact more active than Lipofectamine, even when using 4 times lower siRNA concentrations.

FIG. 31 Splice correction in HeLa cells following treatment of cells with PF3, PF5 or a mix thereof. When using a mix of PF3 and PF5, splicing was increased by almost a factor 4 compared to using either peptide alone at the same molar amount.

### DETAILED DESCRIPTION

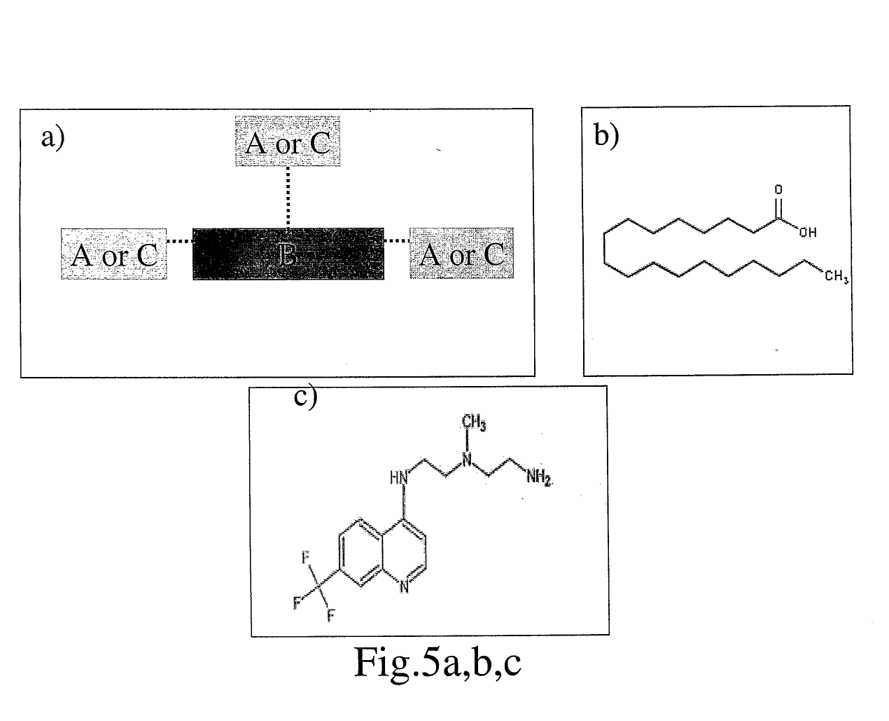

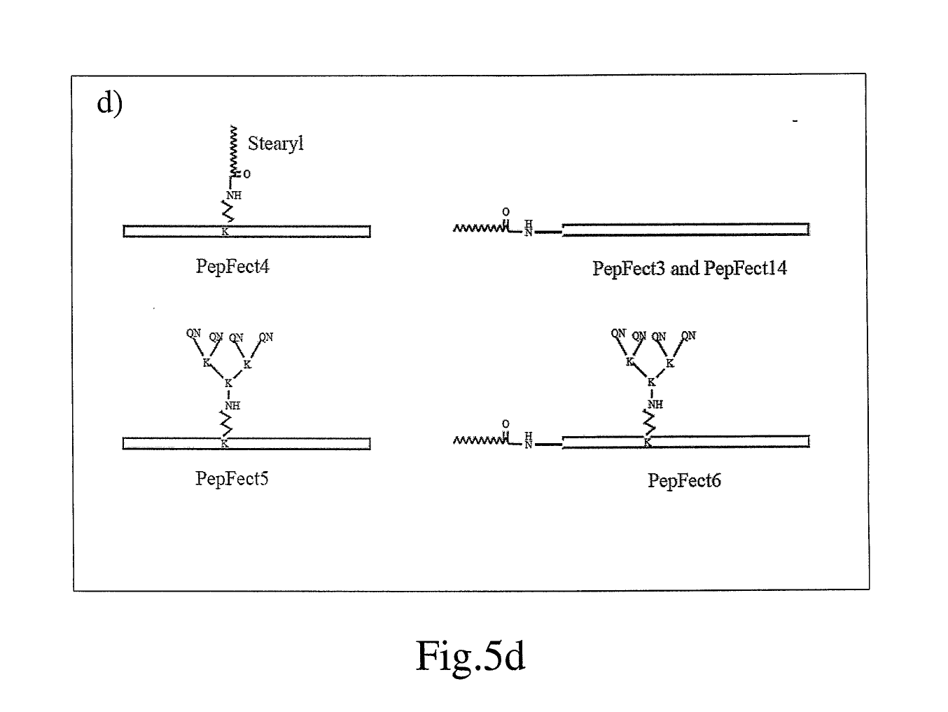

The present invention relates to a system designed for intracellular cargo delivery comprising at least one component A chosen from aliphatic linear or branched moieties with at least 4 carbon and/or cyclic ring systems comprising 2-4 rings which may contain several hetero atoms chosen from N, S, O and P, wherein component(s) A is (are) attached to a cell penetrating peptide B and/or a non-peptide analogue thereof, and in which said delivery system is capable of delivering a cargo by covalent or non-covalent attachment. The delivery system is called PepFect (see examples table 1 and FIG. 5c).

According to one embodiment the delivery system, further comprises at least one component C which is a targeting moiety capable of reaching specific cells or tissue of interest. The targeting moiety may be an aptamer or a targeting peptide such as a homing peptide or a receptor ligand.

According to another embodiment the delivery system further comprises a cargo, which may be delivered into cells, tissue or across a cell layer.

One or more components A, one or more components C and one or more cargos can be coupled covalently either to an amino acid side chain and/or to the N- and/or C-terminal of the peptide (B). In some PepFect compounds, a branched tree-like structured spacer has been applied. The targeting moiety C, may be added non-covalently or through covalent conjugation.

The cell delivery system may comprise more than one peptide B which may be bound to each other through peptide bonds.

Moreover, one or more of the components A, C and the cargo may be attached to one ore more peptides B via a spacer arm.

According to the invention, the delivery system may comprise one ore more components A, one or more peptides B, one or more targeting components C coupled to each other in any order without any cargo. One ore more peptides B may be coupled to one or more components A in any order and without any targeting components C and without any cargoes. These may be delivered for further coupling of cargoes at a later stage. The invention relates to a method of delivering cargoes into a target cell in vivo or in vitro by using such a delivery system.

One or more peptides B and one or more cargoes may be coupled to one or more components A in any order without any targeting components C. One ore more peptides B and one or more cargoes may be coupled to one or more components A and one or more targeting components C in any order.

The invention also relates to novel cell-penetrating peptides as well as the method how to produce the PepFect constructs.

Component A

Component A can be one or several aliphatic linear or branched moieties with at least 4 carbon and/or ring systems comprising 2-4 rings which may contain several hetero atoms chosen from N, S, O and P, wherein component(s) A is(are) attached to a cell penetrating peptide B and/or a non-peptide analogue thereof.

A may also be any Acyl deriving from any organic compound, preferentially a fatty acid, a stearyl, bile acid or its derivatives, cholesteryl, cholic acid, deoxycholate, lithocholate or palmitate.

The aliphatic component A may be 4-30 carbon atoms and may be a fatty acid. Such an aliphatic acid may comprise 4, 5, 6, 7, 8, 9, 10, 11, 12, 13, 14, 15, 16, 17, 18, 19, 20, 21, 22, 23, 24, 25, 26, 27, 28, 29, 30 carbon atoms or any interval created by these figures. It may also be a derivative thereof. The functional group(s) could instead of a carboxylic acid be any of but not limited to —OH, —SH, —NH2, —CHO, COXR wherein X is either O or S, R is any aliphatic or aromatic moiety, or a counter ion in a salt formation like Na+, K+ etc or a halogen. According to one embodiment the fatty acid may comprise 10-30 carbon atoms and can be chosen from stearic acid or a C18 derivate thereof or lauric-, myristic-, palmitic-, arachidic-, and behenic acid, attached to a side chain residue, C- or N-terminally on the cell-penetrating peptide. Moreover the chain may contain saturated/non-saturated bonds.

In addition, the component A, may also be one or more copies of a two-four fused cyclic system of 2 to 4 rings of 3- to 8 membered rings, saturated or non-saturated, possibly comprising one to several hetero atoms in the ring systems chosen from N, S, O, B or P. These may be but not limited to biphenyl, diphenyl ether, amine, sulphide or peri-and/or 'ortho-fused and be chosen from but not limited to quinoline, isoquinoline, quinoxaline, pentalene, naphthalene, heptalene, octalene, norbonane, adamantane, indole, indoline, azulene, benzazepine, acridine, anthracene, biphenylene, triphenylene and benzanthracene and analogues thereof. Such analogues may comprise one or more carboxylic groups and/or one or more additional functional groups such as but not limited to one or more amines, one or more thiols, one or more hydroxyls, one or more esters and one or more aldehydes.

According to one embodiment, four copies of component A may be conjugated on a side chain residue via a lysine branched spacer.

These ring systems may also be substituted e. g with other groups with pH buffering capacity to destabilize endosomes or a as a condensing moiety for nucleotide interactions. Examples of substituents but not limited to, could be one to several primary, secondary and/or tertiary amines, substituted or included in as any aliphatic or aromatic moiety or combinations thereof, also spaced by zero to several atoms in a linear, branched or cyclic fashion or a combination thereof.

Examples are N′-(7-chloroquinolin-4-yl)-N,N-diethyl-pentane-1,4-diamine(chloroquine) or derivatives thereof, di- to tetra ring systems (naphthalene and/or biphenyl connected), 4- to 8 membered rings, one to several hetero atoms in ring systems attached anywhere to the construct described in 1 (FIG. 5)). Another useful example is N-(2-aminoethyl)-N-methyl-N′-[7-(trifluoromethyl)-quinolin-4-yl]ethane-1,2-diamine They may have spacer arm(s) of different lengths (FIG. 5c).

Introduction of a quinoline analogue is accomplished by coupling to activated succinylated side-chains of multiple lysine residues, providing multiple copies of the quinoline analogue covalently bound to the carrier. The preferred conditions are described in Example 12.

The invention also relates to method for synthesizing a quinoline analogue-coupled peptide, or a non-peptide analogue thereof, comprising (a) the steps for activating the lysine on peptide and (b) covalent coupling the quinoline analogue. Activation of the lysine pendant groups of peptide, in order to enable coupling of chloroquine-amine derivative (further disclosed in the Example 12), is achieved by suitable modification of epsilon-amino groups of lysine residues using succinic anhydride. Thus obtained multiple carboxyl groups of the peptide are further activated in situ, i.e. simultaneously with the coupling of quinoline analogue. This procedure is superior to the procedure described in literature up to date, where semi-stable active ester, like NHS, are formed and then coupling hydroxychloroquine, which was derivatised by amine. The method here described is novel and gives better control over the reaction and higher yield. This quinoline analogue is novel, there is no aminoalkylation of chloro-trifluoromethyl-quinoline by a primary diamine derivative in the literature, to our knowledge.

By introducing for example an extra amine at the end of the alkyl chain, covalent attachment is facilitated. The function of the alkyl chain is to provide space and buffer capacity for the aromatic ring system to interact. The chloroquine analogue should consist of but not limited to, quinoline system substituted with a trifluormethylgroup, an alkyl chain with two amines separated with a number of atoms and a functional group at the other end of the alkyl chain separated from the second amine by several atoms for further attachment.

Preferable four copies of component A are conjugated on a side chain via an lysine branched spacer.

The invention further envisages coupling of multiple copies of the quinoline derivative to the peptide B containing appropriate number of poly(L-lysine) pendant groups, all modified by succinic anhydride or any other suitable derivatization reagent known to the skilled in the art.

Peptide Component B

The peptide component B may be selected from one or several copies of the following sequences:

| Sequence | SEQ ID No |
|---|---|
| AGYLLGKINLKALAALAKKIL | 1. |
| AGYLLGKLLOOLAAAALOOLL | 2. |
| RKKRKKKRXRHXRHXRHXR | 3. |
| MVTVLFRRLRIRRACGPPRVRV | 4. |
| RKKRKKK(HXH)4 | 5. |
| LLOOLAAAALOOLL | 6. |
| RQIKIWFQNRRMKWKK | 7. |
| RRRRRRRRR | 8. |
| MVTVLFRRLRIRRACGPPRVRV | 9. |
| GALFLGFLGAAGSTMGAWSQPKKKRKV | 10. |
| FILFILFILGGKHKHKHKHKHK | 11. |
| FILFILFILGKGKHKHKHKHKHK | 12. |
| FILFILFILGKGKHRHKHRHKHR | 13. |
| AGYLLGKINLKALAALAKKIL | 14. |
| GDAPFLDRLRRDQKSLRGRGSTL | 15. |
| PFLDRLRRDQKSLRGRGSTL | 16. |
| PFLNRLRRDQKSLRGRGSTL | 17. |
| PFLDRLRRNQKSLRGRGSTL | 18. |
| PFLNRLRRNQKSLRGRGSTL | 19. |
| PFLNRLRRNLKSLRGRLSTL | 20. |
| PFLDRKRRDQKSLRGRGSTL | 21. |
| RHRHRHHHGGPFLDRLRRDQKSLRGRGSTL | 22. |
| PNNVRRDLDNLHACLNKAKLTVSRMVTSLLEK | 23. |
| PNNVRRDLDNLHAMLNKAKLTVSRMVTSLLEK | 24. |
| PNNVRRDLNNLHAMLNKAKLTVSRMVTSLLQK | 25. |
| PFLNRLRRNLKSLRGRLSTL | 26. |
| PFLNRKRRNLKSLRGRLSTL | 27. |
| INLKALAALAKKIL | 28. |

The peptides may be synthesized with a synthesizing device e.g. on Applied Biosystems stepwise synthesizer model 433A. Amino acids may be assembled by t-Boc chemistry using a 4-methylbenzhydrylamine-polysterene resin (MBHA) to generate amidated C-terminus or by F-moc chemistry on a Rink resin.

Moreover, the peptide B may selected from a peptide that contains a sequence of the form Ny₁-Bx₁-Ny₂-Bx₂-Ny₃, where B is a basic amino acid (such as arg, lys, orn, or his) and N is a neutral aminoacid (such as leu, ile, ala, val, phe, trp, ser, thr, gly, cys, gin, met, pro, tyr) and x and y are integers between 2 and 8.

According to one embodiment the peptide B is selected from LLOOLAAAALOOLL [SEQ ID No 6] and especially AGYLLGKLLOOLAAAALOOLL [SEQ ID No 2] or INLKALAALAKKIL[SEQ ID No28 ] and especially AGYLLGKINLKALAALAKKIL [SEQ ID No 1] and deletions, additions insertions and substitutions of amino acids. The invention also relates to peptides having at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 99,5% homology with these sequences. The invention also relates to sub-fragments of the above mentioned peptides having the same properties.

The peptide B may also be non-peptide analogue of a CPP or a scrambled component of the CPP or of the non-peptide analogue thereof.

The term non-peptide analogue is in the present context employed to describe any amino acid sequence comprising at least one non-coded amino acid and/or having a backbone modification resulting in an amino acid sequence without a peptide linkage, i.e. a CO—NH bond formed between the carboxyl group of one amino acid and the amino group of another amino acid. Examples of non-coded amino acids are D-form amino acids, diamino acids, diphenylalanine, Gly, Pro and Pyr derivates.

Furthermore, the present amino acid sequences may either be amidated or occur as free acids.

A scrambled component B means a peptide with the exact amino acid composition but with completely randomized order. Furthermore, a partly inverted sequence is when two or more of the amino acids of the original sequence have been added in reversed order.

Amino acids can be added, inserted, substituted or deleted from the sequences, also non-natural amino acids, without changing their over all cell-penetrating abilities.

The C-terminus of the cell penetrating peptide B and/or the non-peptide analoge thereof may be modified and be chosen from cysteamine or a thiol containing compound, a linear or branched, cyclic or non-cyclic amine containing compound with preferably one additional functional group such as but not limited to COXR wherein X is O or S, R is any aliphatic or aromatic moiety or, a counter ion in a salt formation like Na, K etc, halogen, —OH, —NHR wherein R is a protective group or any aliphatic or aromatic moiety, —SSR wherein R is a protective/activating group or any aliphatic or aromatic moiety.

The cysteamide group on the C-terminal of entity B, is responsible for the unique property, being activated in serum thereby forming a dimer. This dimerasation reaction is catalyzed by oxidative enzymes present in serum. According to the invention a Cysteamide group of one peptide molecule may interact with the Cysteamide group of another peptide molecule in an oxidation reaction. One such sequence may be AGYLLGKINLKALAALAKKIL-Cysteamide. Such a reaction may lead to the formation of a peptide dimer, by creation of a disulfide bridge between thiol-groups located on two different cysteamide-modified peptides. Hence, the sulfhydryl-groups (—SH) of the cysteamide-modified peptides forms disulfide bonds (S—S-bond, disulfide bridge, C—S—S—C) when exposed to oxidative environment.

The delivery system may comprise at least one peptide B, which may be different or the same peptide. Thus, it may comprise 1, 2, 3, 4, 5, 6, 7, 8, 9, and 10 peptides B. They may be dimers and multiples of CPP and may be cyclic and/or branched.

Component C

The C component is a targeting moiety, such as a ligand for a known or unknown receptor. The substrate may be an aptamer and/or targeting peptide.

An aptamer is a double stranded DNA or single stranded RNA molecule that bind to a specific molecular targets, such as a protein or metabolite.

A targeting peptide is a peptide that binds to specific molecular targets, such as a protein or metabolite, for example a homing peptide. A homing peptide is a peptide sequence which have been selected to bind a certain tissue or cell type, usually by phage display.

In addition, the component C may be another molecule that directs the delivery system to a certain cell type or tissue, well known examples are over-expression of growth factors as tumour targets.

The targeting moiety C may also be non-covalently complexed with the component A and B of the delivery system, as a part of a composition.

Generally, a cell-selective CPP will be useful in the targeted transport of any kind of drug or pharmaceutical substance to a variety of specific eukaryotic and/or prokaryotic cellular targets. A cell-selective transport of such cargo is e.g. envisioned for an improved treatment or prevention of infectious diseases, such as diseases caused by a viral, bacterial or parasital infection.

In yet a further embodiment of the present invention, a cell-penetrating peptide and/or a non-peptide analogue thereof is provided that will enter selectively into a certain cell type/tissue/organ, or that transports a cargo that will only be activated in a certain cell type, tissue, or organ type.

We have shown that adding a moiety for targeting the delivery system to a specific cell type or tissue, does not abolish the delivery properties. As seen by Pepfect 7 (ortogonally SA and with CREKA N-term) can deliver both SCO and plasmid in HeLa and CHO cells (data not shown).

Spacer

Spacers may be used for the attachment of component A, C and the cargoe to the component B.

According to one embodiment the spacer comprises one or more amino acids e.g. lysine units, such as 1, 2, 3, 4, 5, 6, 7, 8, 9, 10 amino acids. e.g. lysine units, which may be straight or branched and modified with functional groups or extra carbon atoms for further attachment.

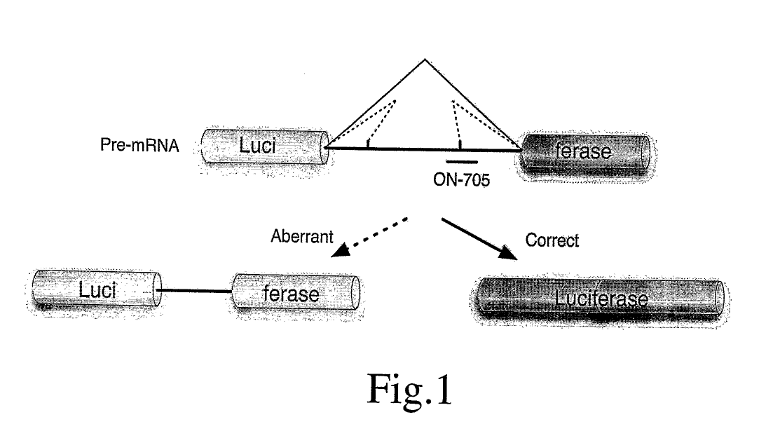

The spacer may be a linear or branched moiety with one to several substituents facilitating attachment of component A or a tree-like structure comprised of preferably but not limited to Lysine or Ornithine residues arranged in a dendrimeric fashion comprising of 1, 2, 3, 4 or unlimited number of Lys or Orn residues as depicted in an example in FIG. 1. The Tree may have any number of branches and is comprised of any number of branching units such as lysine or ornithine residues (here shown as Lys); “Nic” can be nicotinic acid, benzoic acid, quinolinic acid, naphthalene carboxylic acid, chloroquine or its derivative, or any other organic molecule.

A spacer is preferable used in conjugating four copies of the ring system (component A) via a side chain in component B. The spacer may be a dendrimer.

Dendrimers are repeatedly branched molecules, in this case preferable with a peptide backbone.

Examples of the Pepfect system for intracellular delivery are here presented without cargo, as shown below:

TABLE 1
Sequences of cell-penetrating peptides (CPPs)
including C-terminal modification
according to description above.

| Name | Sequence |
|---|---|
| Pepfect 1 | Stearyl-RKKRKKKRXRHXRHXRHXR-NH₂ |
| Pepfect 2 | Stearyl-MVTVLFRRLRIRRACGPPRVRV-NH₂ |
| Pepfect 3 | Stearyl-AGYLLGKINLKALAALAKKIL-NH₂ |
| Pepfect 4 | AGYLLGK(Stearyl)INLKALAALAKKIL-NH₂ |
| Pepfect 5 | AGYLLGK[KK₂sa₄qn₄]INLKALAALAKKIL-NH₂ |
| Pepfect 6 | Stearyl-AGYLLGK[KK₂sa₄qn₄]INLKALAALAK |
| | KIL-NH₂ |
| Pepfect 7 | CREKA-AGYLLGK(Stearyl)INLKALAALAKK |
| | IL-NH₂ |
| Pepfect 8 | 8a Butyrate-AGYLLGKINLKALAALAKKIL-NH₂ |
| | 8b Hexanoyl-AGYLLGKINLKALAALAKKIL-NH₂ |
| | 8c Salicylate-AGYLLGKINLKALAALAKKIL-NH₂ |
| Pepfect 9 | CREKA-AGYLLGK[KK₂sa₄qn₄]INLKALAALAKK |
| | IL-NH₂ |
| Pepfect 10 | AGYLLGK(Stearyl)IKLKALAALAKKIL |
| Pepfect 11 | AGYLLGK(Stearyl)INLKKLAALAKKIL |
| Pepfect 12 | AGYLLGK(Stearyl)INLKALKALAKKIL |
| Pepfect 13 | (qn₄sa₄K₂K)AGYLLGKINLKALAALAKKIL-NH₂ |
| Pepfect 14 | Stearyl-LLOOLAAAALOOLL-NH₂ |
| | RKKRKKK(HYH)₄-NH₂ |
| NucRBut | RKKRKKKRXRHXRHXRHXR-NH₂ |
| TP10 | AGYLLGKINLKALAALAKKIL-NH₂ |
| Pen | RQIKIWFQNRRMKWKK-NH₂ |
| Arg9 | RRRRRRRRR-NH₂ |
| M918 | MVTVLFRRLRIRRACGPPRVRV-NH₂ |
| Stearyl-Arg9 | Stearyl-RRRRRRRRR-NH₂ |
| TP10-Cya | AGYLLGKINLKALAALAKKIL-Cya |
| MPG | GALFLGFLGAAGSTMGAWSQPKKKRKV-Cya |

X: 4-amino-butanoic acid,
Cya: Cysteamide,
Y: Hexanoic acid,
sa: succinicc acid
qn: novel 2-4 ring systems such as quinoline and naphthalene analogues

According to the present invention a new series of CPPs are provided, especially with stearyl modifications. For example (PepFect 1-4 and 14) can be utilized for efficient delivery of SCOs using a non-covalent approach. It is shown that the PepFect peptides are equally, or even more efficient than the commercial transfection reagent Lipofectamine 2000 in conveying SCOs inside cells, still being less toxic. In addition, the low toxicity of Pepfect 1-4 renders them suitable for transfection in sensitive cell systems where Lipofectamine 2000 is not functional. Furthermore, the PepFect peptides are more potent than conventionally used CPPs and far more potent than the pre-clinically used CPP-conjugate (RXR)4-PMO. In addition, the peptides can be effectively exploited for plasmid transfections, also this difficult task in hard to transfect primary glial cells. Moreover, of great importance, only very low amounts of delivery agent and ONs are needed to gain a biological response, which decrease both labours and costs.

The PepFect5-13 analogs may be further chemically modified. Instead of, or in addition to, being modified with a stearic acid entity, these may also be conjugated to a lysine tree bearing e.g. one or more such as four QN analogs that facilitates release of the ONs from vesicular compartments. These are not only active for transportation of ON compounds acting in the nucleus of cells but can additionally be efficiently utilized for the delivery of cytoplasmically active ONs such as anti-miRs and siRNAs. In fact, both PepFect5 and PepFect6, and in particular the latter peptide is significantly more active than Lipofectamine 2000 for the delivery of siRNAs in various cell types. While Lipofectamine/siRNA complexes rarely generates more than 80% down-regulation of gene expression at any given siRNA concentration, both PepFects complexed with siRNA confers almost complete RNAi at low siRNA concentrations. Furthermore, they transfect entire cell populations and not only dividing cells. Finally, PepFect6 is highly active even in serum containing media and is able to transfect very “difficult to transfect” cells including SHSY-5Y, N2a, Jurkat suspension cells, embryonal fibroblasts and primary glia cells. The above described properties, in combination with the lower toxicity compared to Lipofectamine 2000 or Oligofectamine, makes this particular vector highly unique.

TABLE 2
Table showing which cells that has been
subect for siRNA treatments using PF6

| | | 70% | | 90% | | Compared to |
|---|---|---|---|---|---|---|
| Cells | Target | inhibition | | inhibition | | Lipofectamine |
| HeLa | luciferase | 10-20 | nM | 25-50 | nM | better |
| | HPRT1 | 5-10 | nM | 10-20 | nM | better |
| Bhk21 | Luciferase | 2.5-5 | nM | 5-20 | nM | better |
| U2OS | Luciferase | 5-10 | nM | 10-20 | nM | superior |
| | HPRT1 | nd | | 25 | nM | superior |
| CHO | EGFP | 10 | nM | 20-25 | nM | better |
| N2a | HPRT1 | 25-50 | nM | 50-100 | nM | better |
| SHSY5Y | TACE | nd | | 50-70 | nM | superior |
| | HPRT1 | 10-25 | nM | 50-100 | nM | superior |
| B16 | HPRT1 | nd | | 25-50 | nM | better |
| Primary glia | HPRT1 | nd | | 25-50 | nM | superior |
| Jurkat | HPRT1 | nd | | 100 | nM | superior |
| MEFs | HPRT1 | 10-25 | nM | 25-50 | nM | better |

The Cargo

The cargo may be chosen from gene modulating compounds, such as oligonucleotides or plasmids. They may be attached to the delivery system by covalent attachment or complex formation.

The family of oligonucleotides includes antisense oligonucleotides for mRNA silencing, splice correcting oligonucleotides for manipulation of pre-mRNA splicing patterns, and short interfering RNAs for gene silencing.

The cargo may be selected from the group consisting of oligonucleotides and modified versions thereof, single strand oligonucleotides (DNA, RNA, PNA, LNA and all synthetic oligonucleotides), double-strand oligonucleotides (siRNA, shRNA, decoy dsDNA etc.), plasmids and other varieties thereof, synthetic nucleotide analogs for the purpose of inhibition of viral replication or antiviral ONs.

The delivery system makes it possible to release ONs (as cargoes) at the correct intracellular location without addition of extra chloroquine. Because the attachment of four copies of the ring system A increases the local effect of the chloroquine analogues. This is a valuable property for in vivo applications. Also, by conjugating chloroquine to the peptide, the effective concentration is resuced by more than a log, most likely explaining the lack of toxicity otherwise seen with chloroquine at 100 μM concentrations.

It has been estimated that 20-30% of all disease-causing mutations affects pre-mRNA splicing. Several genetic disorders and other diseases, including β-thalassemia, cystic fibrosis, muscular dystrophies, cancers, and several neurological disorders, are associated with alterations in alternative splicing, reviewed in. The majority of mutations that disrupt splicing is single nucleotide substitutions within the intronic or exonic segments of the classical splice sites. These mutations result in either exon skipping, use of a nearby pseudo 3′- or 5′splice site, or retention of the mutated intron. Mutations can also introduce new splice sites within an exon or intron.

One of the first splicing mutations described was found in β-thalassemia patients, where mutations in intron 2 of β-globin pre-mRNA create an aberrant 5′splice site, concomitantly activating a cryptic 3′splice site. This in turn leads to an intron inclusion and non-functional protein. Same type of mutations has been identified in the cystic fibrosis transmembrane conductance regulatorgene, resulting in aberrant splicing and development of cystic fibrosis. Duchenne muscular dystrophy (DMD), characterized by progressive degenerative myopathy, and its milder allelic disorder, Becker muscular dystrophy (BMD), are both caused by mutations in the dystrophin gene. Most nonsense mutations within this gene result in premature termination of protein synthesis and to the severe DMD, whereas a nonsense mutation within a regulatory sequence generates partial in-frame skipping of an exon and is associated with the milder BMD. Also, several types of cancers are known to emenate from mutations affecting alternative splicing. Thus, by using oligonucleotides that sequence specifically bind to these intronic/exonic mutations, these mutations are masked and splicing restored.

Further, the invention relates to a method of delivering cargoes into a target cell in vivo or in vitro. Formation of the complex between PepFect and the ONs described here (siRNA, plasmid, SCO (splice correcting Ons)) may be carried out in a small volume of sterile water 30 minutes in RT, and then added, in most experiments, in full serum containing media. An example of the complex formation with cargo: Phosphorothioate 2′O methyl RNA (SCO) or anti-miR21 2′OMe RNA may be mixed with CPPs at different molar ratios (1:0-1:20) in MQ water in 1/10^{th} of the final treatment volume (i.e 50 μl). Complexes can be formed for about 30 min in RT. After 30 min, complexes were added to cells grown in 450 μl of fresh serum free media.

The cargo may also be selected from a fluorescent marker, a cell- or a linker comprising a cleavable site coupled to an inactivating peptide, peptide ligands, cytotoxic peptides, bioactive peptides, diagnostic agents, proteins, pharmaceuticals e.g. anticancer drugs, antibiotica, chemotherapeutics.

The cargo may be attached to any of the components A, B and/or C by covalent or non-covalent bonds. According to one embodiment the cargo may be attached to the peptide component B. In one embodiment of the invention, the cell-penetrating peptide may be coupled by a S—S bridge to said cargo. Naturally, there are a broad variety of methods for coupling a cargo to a CPP, selected individually depending on the nature of CPP, cargo and intended use. A mode for coupling can be selected from the group consisting of covalent and non-covalent binding, as biotin-avidin binding, ester linkage, amide bond, antibody bindings, etc.

The anticancer drugs may be an alkylating agent, an antimetabolite and a cytotoxic antibiotic. The alkylating agent may include 4-[4-Bis(2-chloroethyl)amino)phenyl]butyric acid (chlorambucil) or 3-[4-(Bis(2-chloroethyl)amino)phenyl]-L-alanine (Melphalan), the antimetabolite is N-[4-(N-(2,4-Diamino-6-pteridinylmethyl)methylamino)-benzoyl]-glutamic acid (Methotrexate) and the cytotoxic antibiotic is (8S,10S)-10-[(3-Amino-2,3,6-trideoxy-α-L-lyxo-hexopyranosyl)oxy]-8-glycoloyl-7,8,9,10-tetrahydro-6,8,11-trihydroxy-1-methoxy-5,12-naphthacenedione (Doxorubicin).

The delivery system may further comprise at least one imaging agent and/or labelling molecule and/or chemotherapeutics. The delivery system of the invention may then be used as chemotherapeutics and/or imaging agents. Such composition may possibly also comprise targeting sequences. The chemotherapeutics and/or imaging agent may be used for delivery of antiviral oligonucleotides.

The labelling molecules may be molecular beacons, including quenched fluorescence based beacons and FRET technology based beacons, for labelling or quantification of intracellular mRNA.

Molecular beacons are molecules, e.g. single-stranded oligonucleotides, with internal fluorophore and a corresponding quenching moiety organized in a hair-pin structure so that the two moieties are in close proximity. Upon binding a target nucleic acid sequence or exposure to other structural modification, the fluorophore is set apart from the quenching moiety resulting in possibility to detect the fluorophore. The most commonly used molecular beacons are oligonucleotide hybridisation probes used for detection of specific DNA or RNA motifs. Similarly, FRET probes are a pair of fluorescent probes placed in close proximity. Fluorophores are so chosen that the emission spectrum of one overlaps significantly with the excitation spectrum of the other. The energy transferred from the donor fluorophore to the acceptor fluorophore is distance-dependent and therefore FRET-technology based beacons can be used for investigating a variety of biological phenomena that produce changes in molecular proximity of the two fluorophores.

The delivery system may also be conjugated to, or complexed with circulation clearance modifiers, like PEG. Such systems may be used for retarded delivery of cargoes. Circular clearance modifiers are molecules that prolong the half-life of drugs in the body, examples are pegyl, albumin binding or sequence capping.

The delivery system may be used in diagnosis of diseases, as research tool and as a targeting system.

The invention also relates to a composition comprising one or more delivery system as defined herein. In such a composition the delivery systems may comprise different components A, and/or different peptides B, and/or different targeting components C and/or different cargoes. These delivery systems may comprise different combinations of A, B, C and cargo as mentioned above.

The invention also relates to a pharmaceutical composition comprising the delivery system according and/or a composition as defined above.

It also relates to the use of one or more delivery systems for the production of a pharmaceutical composition.

Especially the composition may comprise at least two different delivery systems that may act additative or synergistic. These may be present in the composition in different ratios. For example, the compositions may comprise any combination of the Pepfects disclosed in table 1 e.g. Pepfect 5 and 6.

Such a composition may also comprise a mixture of at least two peptides in the same or in different delivery systems which peptides each bring a different property to the complex, such as targetting and transfection.

Such a pharmaceutical composition may be in the form of a oral dosage unit; an injectable fluid; a suppository; a gel; and a cream and may comprise excipients, lubricants, binders, disintegrating agents, solubilizers, suspending agents, isotonizing agents, buffers, soothing agents, preservatives, antioxidants, colorants, sweeteners.

For example, the delivery agent may also be used as an antimicrobial composition, as cell-penetrating peptides resembling those of lytic peptides.

The invention also relates to a material covered with one or more of the delivery systems according to the invention.

Further, it relates to a material having one or more of the delivery systems according to any of claims 1-16 incorporated into the material. The delivery system according to the invention may be incorporated into the dendrimers, liposomes etc. Liposomes are composite structures made of phospholipids and may contain small amounts of other molecules

The invention also relates to a novel peptide that contains a sequence of the form Ny₁-Bx₁-Ny₂-Bx₂-Ny₃, where B is a basic amino acid (such as arg,lys,orn, or his) and N is a neutral aminoacid (such as leu, ile, ala, val, phe, trp, ser, thr, gly, cys, gln, met, pro, tyr) and x and y are integers between 2 and 8.

The invention especially relates to peptides wherein the entity B is selected from LLOOLAAAALOOLL [SEQ ID No 6] and especially AGYLLGKLLOOLAAAALOOLL[SEQ ID No2] or INLKALAALAKKIL [SEQ ID No28] and especially AGYLLGKINLKALAALAKKIL [SEQ ID No 1] and the sequences with deletions, additions insertions and substitutions of amino acids. The invention also relates to peptides having at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% homology with these sequences. The invention also relates to sub-fragments of the above mentioned peptides having the same properties.

As described previously, a CPP can be coupled to a cargo to function as a carrier of said cargo into cells, various cellular compartments, tissue or organs. The cargo may be selected from the group consisting of any pharmacologically interesting substance, such as a peptide, polypeptide, protein, small molecular substance, drug, mononucleotide, oligonucleotide, polynucleotide, antisense molecule, double stranded as well as single stranded DNA, RNA and/or any artificial or partly artificial nucleic acid, e.g. PNA, a low molecular weight molecule, saccharid, plasmid, antibiotic substance, cytotoxic and/or antiviral agent. Furthermore, the transport of cargo can be useful as a research tool for delivering e.g. tags and markers as well as for changing membrane potentials and/or properties, the cargo may e.g. be a marker molecule, such as biotin.

With respect to the intended transport of a cargo across the blood-brain barrier, both intracellular and extracellular substances are equally preferred cargo.

All specific details relate mutatis mutandis to all embodiments described herein. When for example specific chemical components are described in relation to the components A, B, and C of the delivery system, these also applies when the delivery system is incorporated into the dendrimers or liposomes, a material covered with the delivery system as well as when a delivery system it is covered with circulation clearance modifiers.

The invention will now be further illustrated by the following description of embodiments, including short description of the drawings, materials and methods, examples including figures and figure legends as well as sequence listing, but it should be understood that the scope of the invention is not limited to any specifically mentioned embodiments or details.

### EXAMPLES & EXPERIMENTS

The invention is below described by examples and comparisons with different PepFect systems and also with Lipofectamine 2000, which is market leading for transfections in vitro today. First the improvements and remodeling of the delivery system are described. Then the testing of various methods to illustrate how versatile PepFects are for delivery of splice correcting ONs, plasmid as well as miRNA and siRNA is being described. In addition, the overall improvement of PepFect compared to Lipofectamine 2000, which have been applied according to the manufacturers instruction, is shown by lower toxicity, homogeneous transfections and high yield transfections in “hard to transfect cells”. All the experiments have been performed in more than one cell type and in most cases with serum present.

### Materials and Methods

Synthesis of Peptides and Oligonucleotides

The peptides in PepFect 1, PepFect 2, PepFect 3, PepFect 4, penetratin [10], TP10 [11], M918 [12], Arg9 [13], and stearyl-Arg9 were synthesized on Applied Biosystems stepwise synthesizer model 433A. Peptides were assembled by t-Boc chemistry using a 4-methylbenzhydrylamine-polysterene resin (MBNA) to generate amidated C-terminus. Solid phase peptide synthesis (SPPS) can also be synthesised using standard Fmoc SPPS conditions well-known to those skilled in the art. Peptides used in the present invention are obtained using standard protocols on a SYRO multiple peptide synthesizer (MultiSynTech GmbH). The method involves repeated coupling of Fmoc-protected amino acids from the carboxy terminal end to the N-terminal end of the peptide, assisted by HBTU activating reagent and DIEA as a base component. The polystyrene resin solid support employed is Rink amide resin (preferable substitution level 0.4 to 0.6 milliequivalents per gram of resin). Amino acids were purchased from Neosystem, France and coupled as hydroxybenzotriazole (HOBt) esters. After cleavage of peptides from the resin using HF, synthesis products were purified by reverse phase HPLC Iomega C₁₈ column and analyzed using Perkin Elmer prOTOF™ 2000 MALDI O-TOF Mass Spectrometer. Masses of peptides correlated well with theoretical values. The sequences of the peptides are presented in Table 1.

For the branched spacer: Resin-bound fully protected peptide sequence Fmoc-AGYLLGK(ε-Mtt)INLKALAALAKKIL-Rink (Rink=Rink amide resin, all amino acids are protected by standard protecting groups if not stated otherwise) is used as starting material. The general manual procedure (Step 1 to Step 7) is to be followed to obtain branched structure of four free carboxyl groups (designated as point of attachment for four copies of novel QN analogues). After each step qualitative ninhydrine colour test (Kaiser test) is performed to monitor the completeness of reaction.

1. The peptide resin is treated with 35% piperidine for 40 minutes to achieve deprotection of amino group.

2. Stearic acid is coupled (the preferred method is use of DCM as solvent and BOP/DIEA for activation and coupling for 1 hour).

3. For Mtt removal, repeated washes by 1% TFA, 3-4% TIS, DCM are employed (1-1.5 hours of total treatment). In order to insure the completeness of removal 1% TFA in DCM without addition of TIS is added to monitor the yellow color of leaving trityl group.

4. 3-5 equivalents of Fmoc-Lys(Fmoc)-OH is coupled for 45 minutes. Preferred coupling reagents are BOP (even more preferably, its non-cancerogenic analog PyBOP) or DIPCDI/HOAt.

5. Fmoc removal according to Step 1.

6. Repeat steps 4 and 5.

7. Coupling of 1.5 equivalents of succinic anhydride in DMF in the presence of 3 equivalents of DIEA for 10 minutes.

Phosphorothioate 2′-O-methyl RNA oligonucleotides were synthesized on an ÄKTA™ oligopilot™ plus 10 with Oligosynt™ 15, pre-packed synthesis columns. Phosphoroamidites (ChemGenes Corporation, Boston, Mass.) at 0.1 M concentration were added in 5 equivalents excess and the recycle time of coupling reagents was 5 minutes. Thiolation was performed with 0.2 M bis-phenylacetyl disulfide (ISIS Pharmaceuticals, Carlsbad, Calif.) in 3-picoline/acetonitrile (1:1) during 3 minutes using 3 ml per synthesis cycle. Oligonucleotides were cleaved from the solid support and deprotected overnight in 25% aqueous ammonium hydroxide (Merck, Darmstadt, Germany) at 55° C. Purification was made with a Tricorn™ column packed with Source™ 15Q anion exchange media utilizing an ÄKTAexplorer™ 100 system and basic NaCl buffers. HiTrap™ desalting columns were used for subsequent work-up of purified oligonucleotides followed by HPLC analysis (Agilent 1100, Santa Clara, Calif.) utilizing a DNAPac™-100 analysis column (Dionex, Sunnyvale, Calif.) confirming >97% full length purity. Correct product was confirmed by mass analysis on a Finnigan™ LCQ™ Deca XP plus mass spectrometer (ThermoFischer Scientific, Waltham, Mass.).

Cell Culture

HeLa pLuc 705 cells, kindly provided by R. Kole and B. Leblue, and hek 293 cells were grown in Dulbecco's Modified Eagle's Media (DMEM) with glutamax supplemented with 0.1 mM non-essential amino acids, 1.0 mM sodium pyruvate, 10% FBS, 100 U/ml penicillin, 100 mg/ml streptomycin and 200 μg/ml hygromycin. CHO cells were grown in DMEM-F12 media with glutamax supplemented with 0.1 mM non-essential amino acids, 1.0 mM sodium pyruvate, 10% FBS, 100 U/ml penicillin, and 100 mg/ml streptomycin. BHK 21 cells were grown in GMEM +2 mM Glutamine +5% Tryptose Phosphate Broth +5-10% Fetal Bovine Serum. Cells were grown at 37° C. in 5% CO₂ atmosphere. All media and chemicals were purchased from Invitrogen (Sweden).

Complex Formation

Formation of ON/CPP complexes: Phosphorothioate 2′O methyl RNA (SCO) or anti-miR21 2′OMe RNA (with 33% LNA substitution) was mixed with CPPs at different molar ratios (1:0-1:20) in MQ water in 1/10^{th} of the final treatment volume (i.e 50 μl). Complexes were formed for 30 min in RT and meanwhile media was replaced in 24-well plates to fresh serum free DMEM (450 μl). Thereafter, complexes were added to each well. The final concentration of SCO was kept constant at 200 nM and peptide concentration was varied or complexes were formed at a given molar ratio using 400 nM SCO and then serial diluted in water. Complexes were prepared according to manufacturers protocol when using the commercial transfection reagent Lipofectamine 2000 (Promega, USA). PepFect/siRNA complexes were essentially formed in a similar manner but using 100 nM siRNA as starting concentrations and molar ratios ranging from 20-40. Lower concentrations were generated by doing serial dilutions.

Formation of plasmid/CPP complexes:0.5 μg of pGL3 luciferase expressing plasmid or pEGFP-C1 plasmid was mixed with CPPs at different charge ratios (1:1-1:4) in MQ water in 1/10^{th} of the final treatment volume (50 μl). After 30 min, complexes were added to cells grown in 450 μl of fresh serum free media. When using Lipofectamine 2000, complexes were prepared according to manufacturers protocol (Promega, USA).

Gel Shift Assay

Peptides were mixed with SCO as previously described. 0.5 μg SCO was mixed with increasing concentrations of peptides giving rise to peptide/SCO molar ratios ranging from 5 to 20. Complexes were analyzed by electrophoresis on a 20% polyacrylamidic gel at 150V for 1 h in TBE buffer, containing ethidium bromide (Sigma, Sweden). Pictures were taken in Fujifilm LAS-1000 Intelligent Dark box II using IR LAS-1000 Lite v1.2 software.

Quantitative Uptake 100 000 HeLa pLuc 705 cells, seeded 24 h prior experiment, were treated with 200 nM Cy5 labeled SCO complexed with peptides at molar ratios for 1 h. After treatment, cells were washed twice in HKR before trypsination. Cells were centrifuged at 1000 g for 5 min at 4° C. and cell pellets were lysed with 250 μl 0.1 M NaOH for 30 min after which 200 μl lysate was transferred to a black 96-well plate. Fluorescence was measured at 643/670 nm on a Spectra Max Gemeni XS fluorometer (Molecular devices, USA) and recalculated to amount of internalized compound by using the linearity of fluorescein and normalizing to amount of protein (Lowry BioRad, USA).

Luciferase Assay

Splice correction experiments: 100 000 HeLa pLuc 705 cells were seeded 24 h prior experiments in 24-well plates in all experiments. Cells were treated for 4 h with complexes in serum free media followed by replacement to serum medium for additionally 20 h. Thereafter, the cells were washed twice with Hepes Krebs Ringer (HKR) buffer and lysed using 100 μl 0.1% triton X100 in HKR for 15 min at room temperature. Luciferase activity was measured on Flexstation II (Molecular devices, USA) using Promega luciferase assay system. RLU values were normalized to protein content and results are displayed as RLU/mg or as fold-increase in splicing over untreated cells. In experiments with the agent promoting endosomal escape, chloroquine, complexes were co-incubated for 2 h with 75 μM chloroquine in serum free DMEM and subsequently the cells were grown for 20 h in complete DMEM.

Plasmid transfections: For plasmid transfection, 80 000 CHO or Jurkat cells were seeded 24 h before treatment. Cells were treated for 4 h with pGL3/CPP complexes in serum free DMEMF12 after which media was replaced for full growth media for additionally 20 h. Cells were lysed and analyzed in accordance with the splice correction assay.

siRNA transfections: Different luciferase-stable cell lines including HeLa, BHK21, and U2OS cells were seeded as in the other experiments. Cells were treated for 4 h in serum-free or full growth media after which 1 ml of full growth media was added to the wells. Cells were lysed and measured for luminescence 20 h later as described previously. The luminescence was normalized to protein content in each well and the RLU/mg value from untreated cells were considered as 100%. Different treatments are then presented as % of untreated cells.

MicroRNA-21 assay: A plasmid, psi-CHECK2, carrying one internal firefly luciferase gene and a second renilla luciferase gene carrying a microRNA-21 target site in the 3′UTR, was transfected into HeLa cells grown in a 6 cm dish. One day after transfection, cells were detached by trypsination and seeded at a density of 70 000 cells/well in a 24-well plate. After another 24 h, cells were treated with antimiR-21 complexes as previously described for SCOs. Cells were lysed and then assessed for fire fly luciferase expression, that act as an internal standard for transfection, and then for renilla expression. If an ON reaches the cytoplasm, miR21 is sequestered and an increase in renilla expression is expected, generating a positive read-out similar to that of the splice correction assay. The firefly/renilla luciferase ratio of untreated cells was set to 1 and increases in renilla after treatment are presented as fold-increases compared to untreated cells. HeLa cells were used since they are known to express high levels of miR21.

Toxicity Measurements

Membrane integrity was measured using the Promega Cytox-ONE™ assay. In brief, 10⁴ cells were seeded in 96-well plates two days before treatment with peptides in serum free DMEM. After 30 min, media was transferred to a black fluorescence plate and incubated for 10 min with Cytolox-ONE™ reagent followed by stop solution. Fluorescence was measured at 560/590 nm. Untreated cells were defined as zero and LDH released by lysating in 0.18% triton in HKR as 100% leakage.

Wst-1 Assay

Long-term toxic effects of peptides were evaluated using the Wst-1 proliferation assay. HeLa pLuc 705 cells were seeded onto 96-well plates, 10⁴ cells/well, two days before treatment. Cells were treated with complexes in 100 μl serum or serum free DMEM for 24 h. Cells were then exposed to Wst-1 according to manufacturers protocol (Sigma, Sweden). Absorbance (450-690 nm) was measured on absorbance reader Digiscan (Labvision, Sweden). Untreated cells are defined as 100% viable.

FACS

A 24-well plate with EGFP-stable CHO cells were seeded one day prior transfection. On the day of transfection, transfect the cells with PF6-siRNA complexes, molar ratios between siRNA:PF6 a) for serum free media incubations: 1:20 and 1:40 with siRNA concentrations 50 nM, 25 nM and 12.5 nM; nd 1:80 with siRNA concentrations 20 nM, 10 nM and 5 nM. b) for full media incubations: 1:20 and 1:40 with siRNA concentrations 100 nM, 50 nM and 25 nM. For control experiments the cells were transfected with EGFP siRNA using Lipofectamine (100 nM siRNA and 2.8 ul of Lipofectamine), or mock transfected without complexes or only with siRNA (to get native EGFP levels). The incubation vol. during transfections was 500 ul. Incubate the cells with the complexes in serum free media or in full media, as required, for 4 hours. Then add 1 ml of full media into each well and let the cells grow for 24 (or, alternatively, for 48 hours).

On the day of measurment, wash the cells with PBS, and detach from the wells by trypsination. Use 125 ul trypsin solution. After the cells have detached, add 500 ul full media and transfer the cells into eppendorf tubes. Centrifuge at 500×g for 10 min, remove supernatant and resuspend in 500 ul of PBS supplemented with 2% FBS (this FBS is quite important, keeps the cells in better shape when the tubes are waiting to be analyzed). Transfer the cell suspensions info FACS tubes and put them on ice. Analyze as soon as possible. Measure the cell suspensions in FACS. Gate the living cell population on FSC-SSC plot. On the FSC/FITC-A or SSC/FITC-A plot find the gates for moctransfected cells (gate/cloud for cells with native EGFP levels). Run the samples using the same gates. All the cells falling out from the cloud with native EGFP-levels and having lower EGFP levels will be counted as cells where the siRNA transfection has taken place. Present the results as % of cells transfected with siRNA. Alternatively, choose the gates in a similar way based on mock-transfected cells using FITC-A histogram graph.

### EXAMPLE 1

### (FIGS. 1-3): Splice Correction by Cell-Penetrating Peptides

In order to evaluate the potency of the different unmodified CPPs to convey oligonucleotides into cells, we used a so-called splice correction assay [14]. This assay is based on a stably transfected HeLa cell line, HeLa pLuc 705 cells. The cells have a stable transfection of a plasmid carrying the luciferase coding sequence, interrupted by an insertion of intron 2 from β-globin pre-mRNA carrying a cryptic splice site (FIG. 1). Under normal conditions, the cryptic splice site will activate an aberrant splice site giving rise to a mature mRNA with an intron inclusion and, thus, a non-functional luciferase protein. However, if the aberrant splice site is masked by an SCO, the pre-mRNA of luciferase will be properly processed and functional luciferase produced. By using the HeLa pLuc 705 cells, various vector efficiencies in nuclear delivery can be evaluated by measuring the luciferase activity.

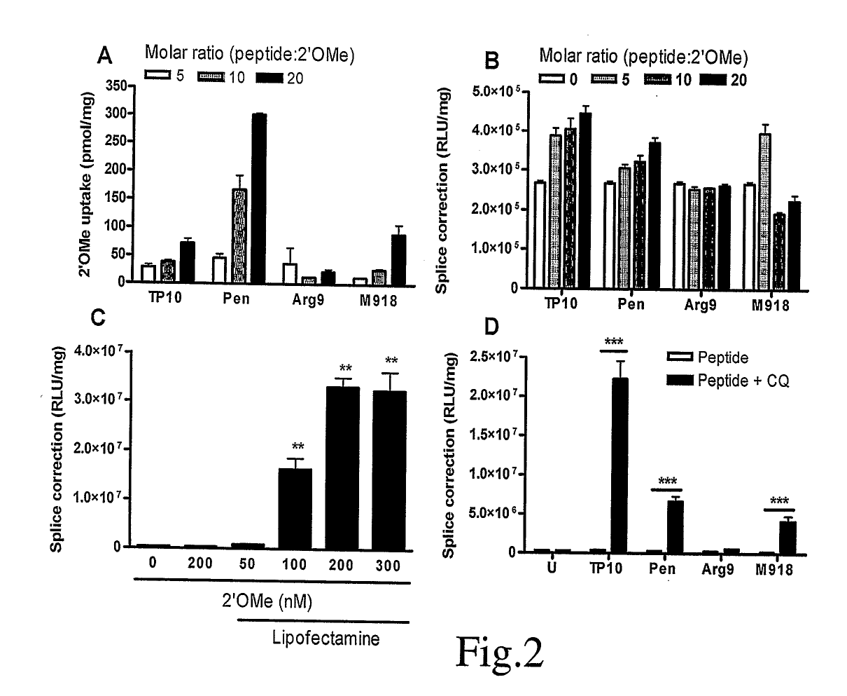

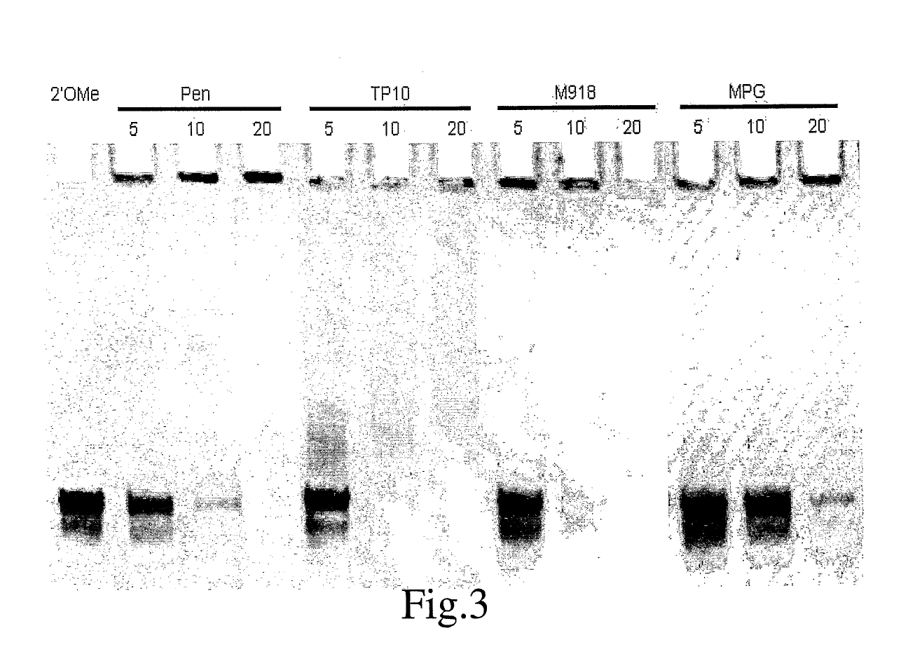

In the first set of experiments we wanted to evaluate the ability of several established CPPs to deliver splice correcting phosphorothioate 2′O-methyl RNA (i.e SCO) into cells using the co-incubation strategy [15]. As seen in FIG. 2a, all tested unmodified CPPs were able to translocate into cells within 1 h of exposure, penetratin being the most efficient peptide. However, none of the peptides were capable of conveying bioactive SCOs into the nucleolus of the cells, interpreted from the insignificant increase in luciferase expression (FIG. 2b). In order to confirm that the negative results were not a result of inactive SCO, a control experiment was conducted using Lipofectamine 2000 as a delivery vector. As seen in FIG. 2c, a dose-dependent increase in correctly spliced luciferase was observed with increasing SCO concentration. Another possible reason for the low activity could be that complexes between CPPs and SCOs were unable to form. However, also this was ruled out as all peptides promoted complex formation in a gel retardation assay (FIG. 3). The results collectively suggest that albeit the CPPs are able to convey the SCO inside cells, they most likely remain trapped in endosomal compartments. This was further supported by confocal microscopy data where a large amount of, if not all, labelled SCOs were residing in punctuate compartments, eg. endosomes or lysosomes (data not shown). More important, when co-treating cells with the lysosomotrophic agent chloroquine, splice correction was increased significantly with all peptides except Arg9 (FIG. 2d). Interestingly, TP10 was superior to the other peptides even though penetratin generated the highest quantitative uptake. Taken together, this suggests that the co-incubation strategy promote cellular uptake, but not bioavailable delivery. Furthermore, it was concluded that high cellular uptake does not correlate with high activity of the transported cargo.

### Example 2

### (FIGS. 2, 4, 6): Bifunctional CPPs to Facilitate Splice Correction

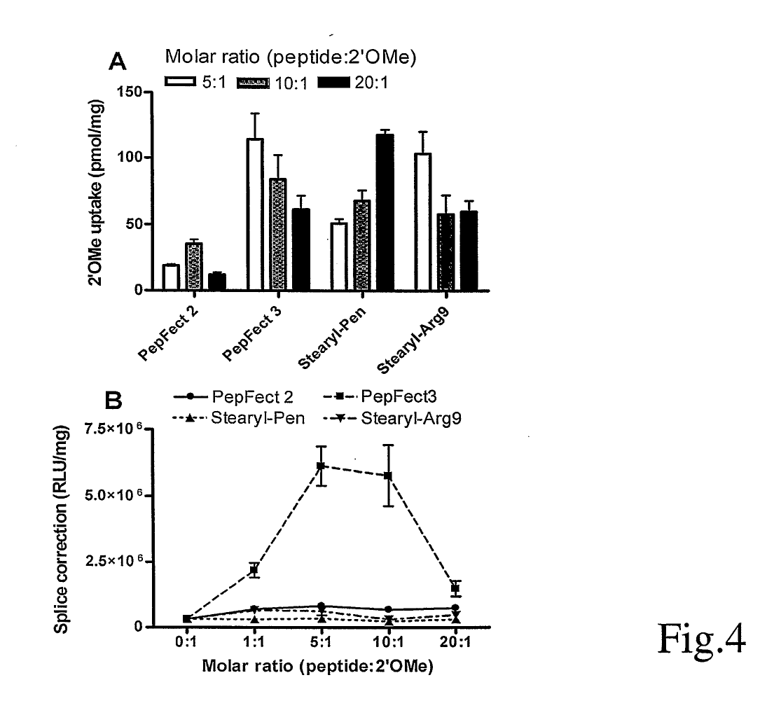

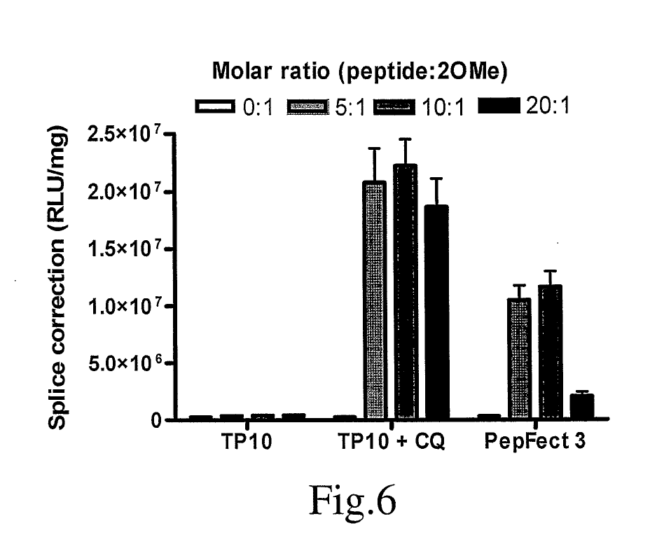

Next, modifications were introduced in the CPPs in an attempt to increase their efficiency. A fatty acid moiety (i.e stearic acid) was introduced to existing CPPs and also to a newly designed sequence (see FIG. 5.). The rational behind the design of the latter peptide, PepFect 1, was to create a bifunctional peptide, with one moiety for promoting endosomal escape (i.e (RHbutRH)₄) and one part to confer nucleolar delivery (RKKRKKK). Although the peptide was highly potent in transfection with peptide nucleic acids as a covalent conjugate, it was not capable of transporting SCOs in a non-covalent setting (data not shown). Therefore the peptide was N-terminally stearylated. In parallel, the other four previously tested CPPs were also stearylated, and M918 [12] and TP10 [11] renamed PepFect 2 and PepFect 3, respectively. Interestingly, neither stearylated Arg9 nor penetratin was able to promote splice correction at any molar ratio tested. However, PepFect 3 was extremely potent at molar ratios up to 10:1 over SCO, reaching almost the same level of luciferase expression as when using Lipofectamine 2000 (FIG. 4). In the case of PepFect 3, there was a direct correlation between uptake and splice correction, while for the other peptides there were no such correlation. In fact, the splice correction was almost as high using PepFect 3 as when using TP10 together with chloroquine (FIG. 6). Since the difference in quantitative uptake was insignificant between TP10 and PepFect 3 in complex with SCO (FIG. 2a and FIG. 4a), it was concluded that the increased activity is a result of increased endosomal release. Surprisingly, only PepFect 3 was active in the splice correction assay and PepFect 2 only had minor activity (FIG. 4b).

An increasing number of diseases, such as β-thalassemia, cystic fibrosis, muscular dystrophies, cancer etc, are caused by mutations leading to aberrant splicing which now may be restored using different SCOs [3,17,18]. However, high doses of SCOs are needed in order to attain significant biological effects in vivo. Therefore, these results are extremely promising for future treatment of various diseases emanating from defective alternative splicing.

### Example 3

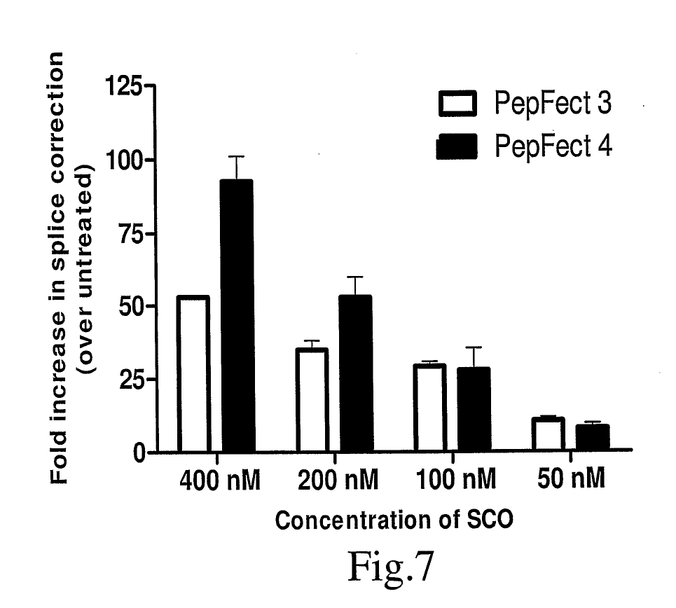

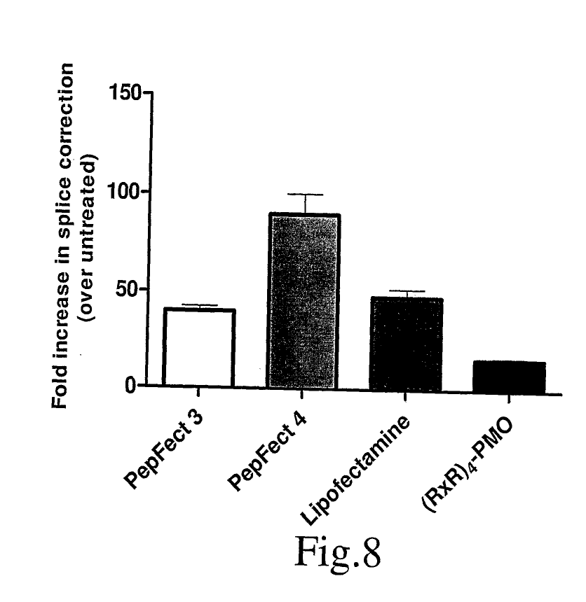

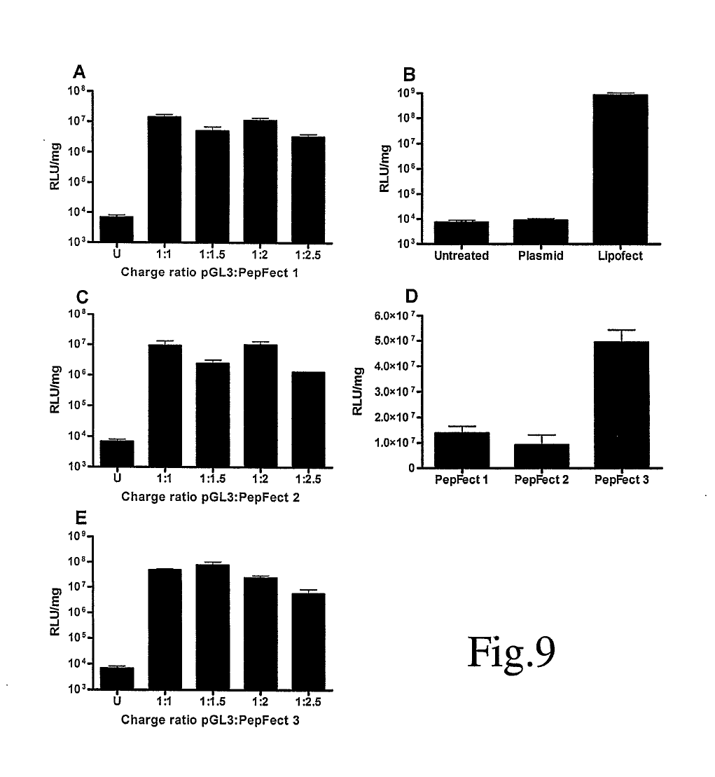

### (FIGS. 7-8): Evaluation of Position of Fatty Acid Modification

To evaluate and characterise the position of the fatty acid modification PepFect 3 (Stearyl modification at N-terminal) and PepFect4 (side chain stearylated on Lys7) where compared in efficiency to deliver SCO. FIG. 7 shows that PepFect 4 is more potent than PepFect 3 in promoting splice correction, even at a lower molar ratio. Also, in comparison with Lipofecatmine 2000, PepFect4 is more potent. Interestingly, both PepFetcs are significantly more active than the pre-clinically used covalent CPP-morpholino conjugate, RXR4-PMO at 25 times lower SCO concentration (FIG. 8.).

### Example 4

### (FIGS. 9-12): Plasmid Delivery with PepFect 3 and PepFect4

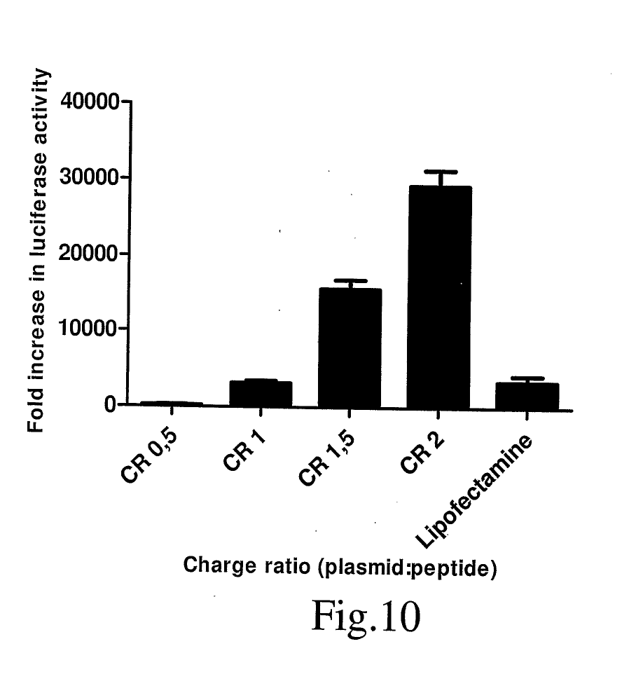

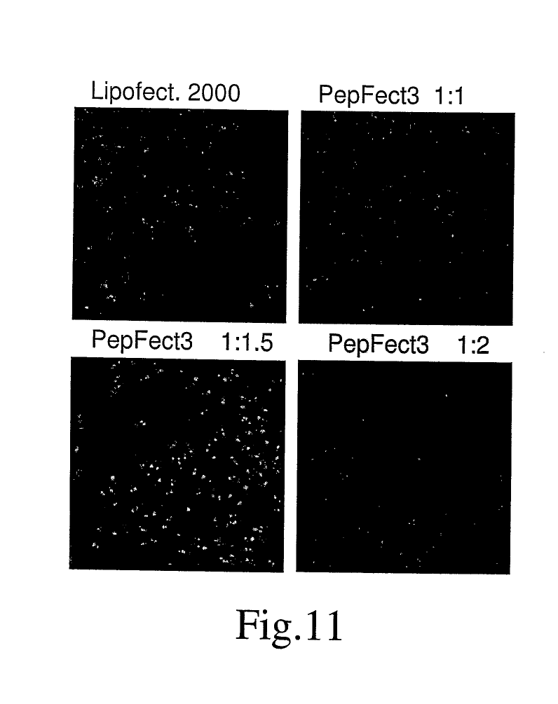

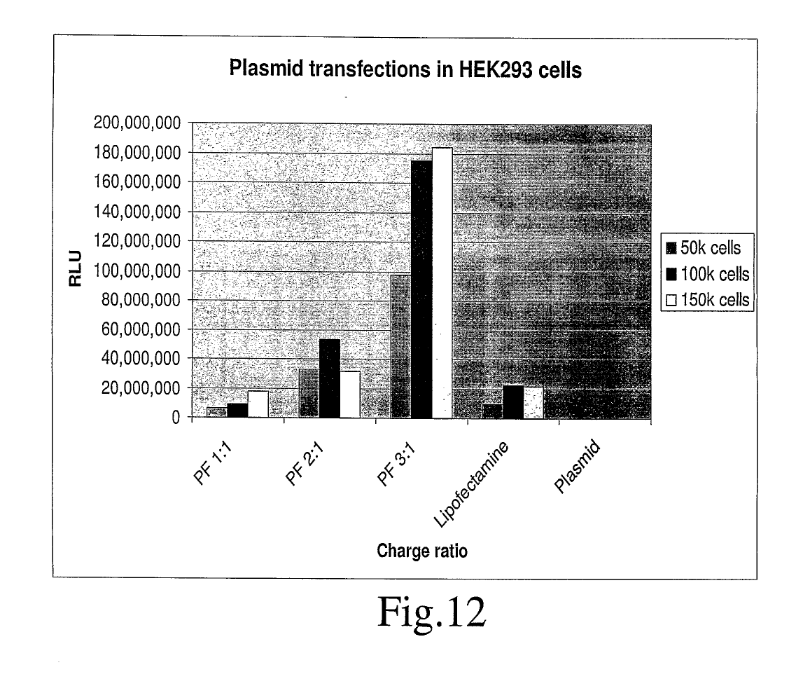

In the next set of experiments, the ability of the above mentioned PepFect peptides to promote uptake and expression of a 4.7 kbp luciferase expressing pGL3 plasmid was evaluated. Surprisingly, all PepFect peptides were able to significantly increase gene delivery (FIG. 9.) Again, PepFect 3 was the most effective peptide, at a charge ratio between 1:1 and 1:1.5. The results suggest that the peptides could be effectively utilized for gene delivery also of more biologically relevant plasmids and ONs. Complexes of PF3 and plasmid were prepared at different charge ratios ranging from CR 0.5-2 and compared to the transfection efficiency of Lipofectamine 2000 applied according to manufacturers protocol. Results are presented as fold increase in gene expression compared to cells treated with plasmid only. The graph in FIG. 10 illustrates that at CRs above 1, PF3 is more active than Lipofectamine 2000. An additional advantage is the homogeneous transfection as seen in FIG. 11. Traditional transfection reagents need a certain cell confluency to function optimal however, FIG. 12 shows that PF4 transfect equally well independent of the cell number seeded.

### Example 5

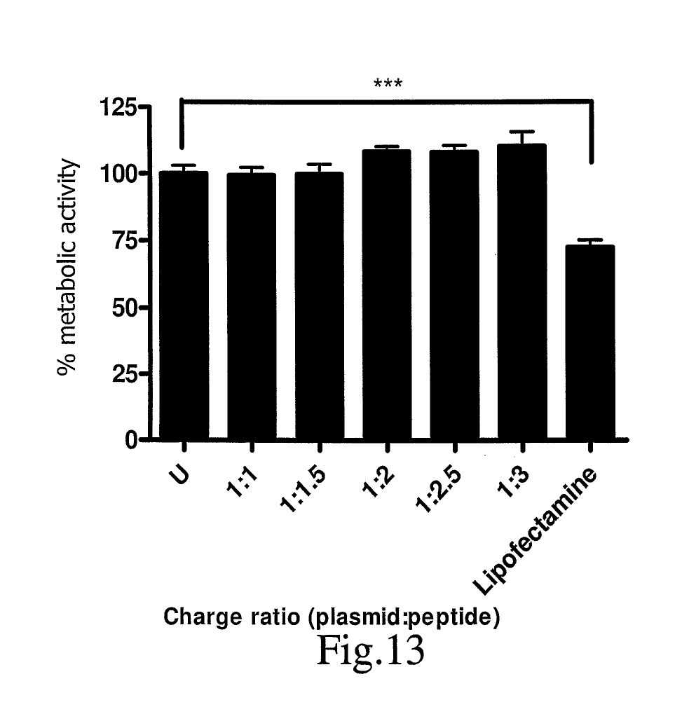

### (FIGS. 11 and 13): Pepfect is Less Cytotoxic than LF 2000

Importantly when working with living tissue, is to keep the toxic effects as low as possible. As seen in FIG. 11, the same number of cells was seeded in the different samples before the transfection, however, after treatment the number of cells are lower in the lipofectamine treated cells. PepFect 3 is less cytotoxic than Lipofectamine 2000 (FIG. 13) and may therefore offer a new non-viral tool for gene therapy.

### Example 6

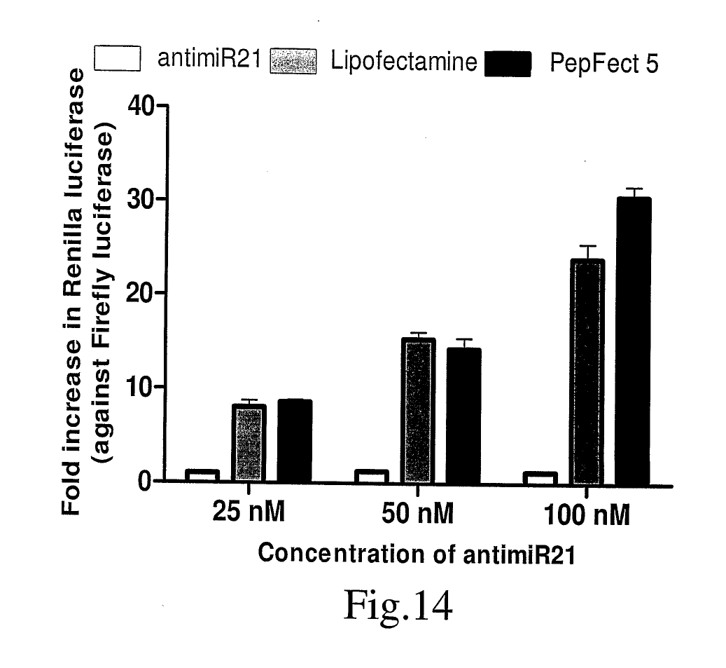

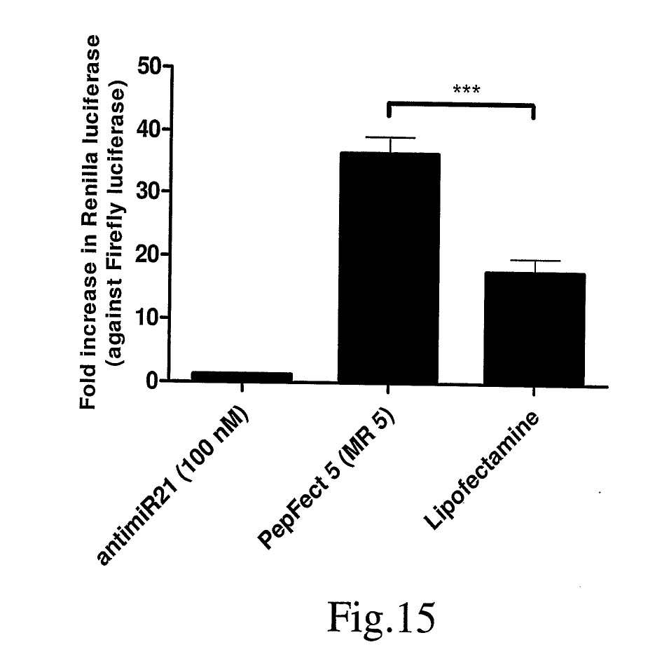

### (FIGS. 14-15): MicroRNA Delivery by PepFect 5

MicroRNAs (miRNAs) represent a new class of noncoding RNAs encoded in the genomes of plants, invertebrates, and vertebrates. MicroRNAs regulate translation and stability of target mRNAs based on (partial) sequence complementarity. miRNA alterations are involved in the initiation and progression of human cancer. It has been shown that miR-21 functions as an oncogene and modulates tumorigenesis through regulation of genes such as bcl-2 and thus, it may serve as a novel therapeutic target [19].

Here we show miR-21 delivery by PepFect 5 as compared to Lipofectamine (FIG. 14). Both are equally efficient at a lower ON molar ratio with PepFects 5. However, at a molar ratio of 5, Pepfect 5 is significantly better (FIG. 15).

### Example 7

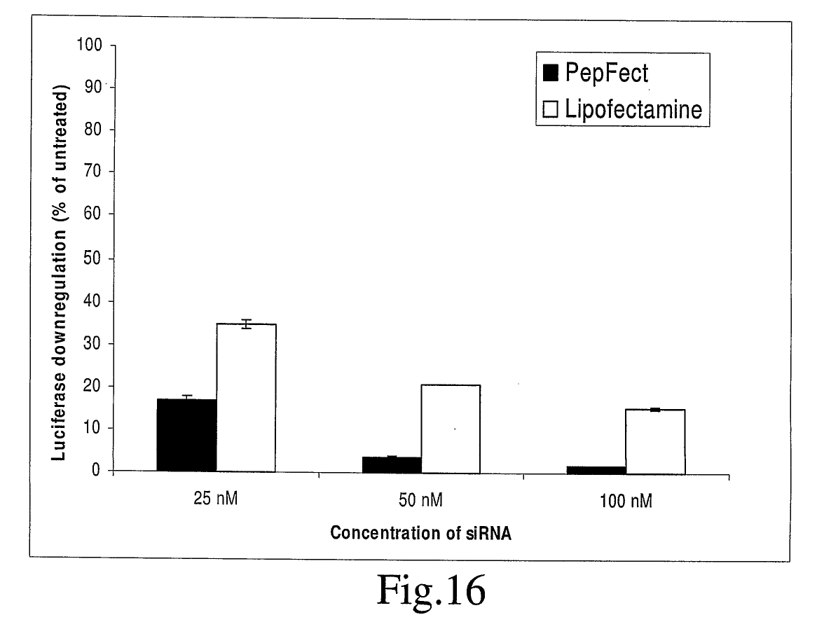

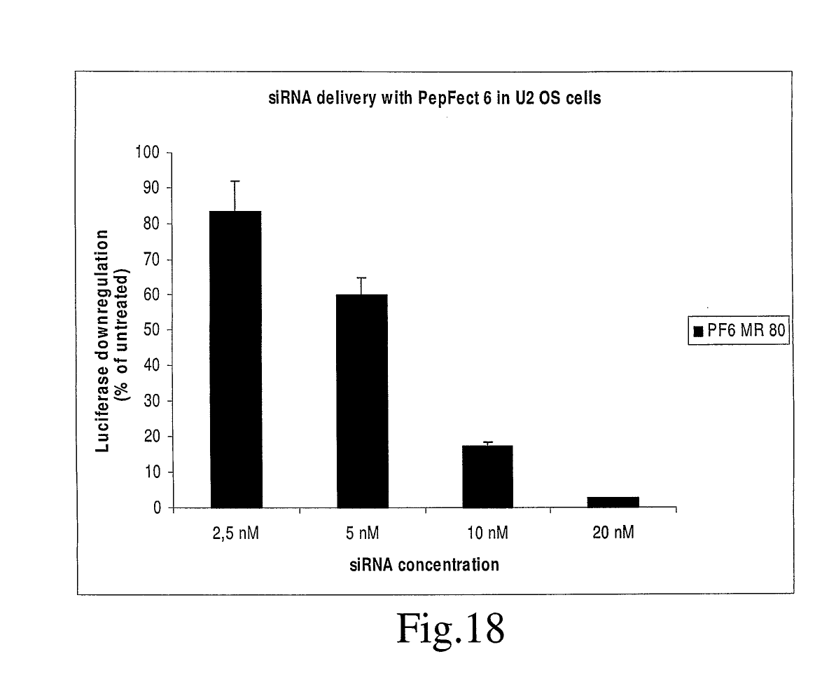

### (FIGS. 16-19): Comparison of PepFect5 and PepFect6 for siRNA Delivery

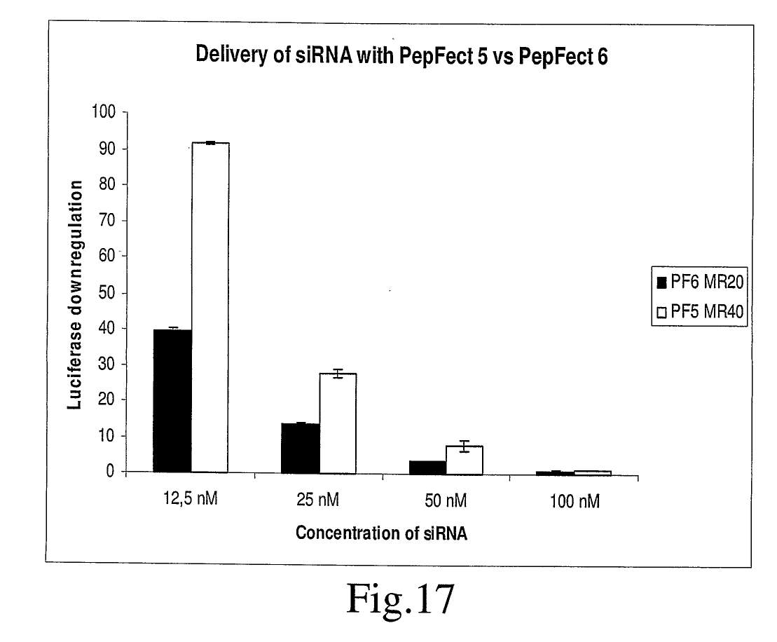

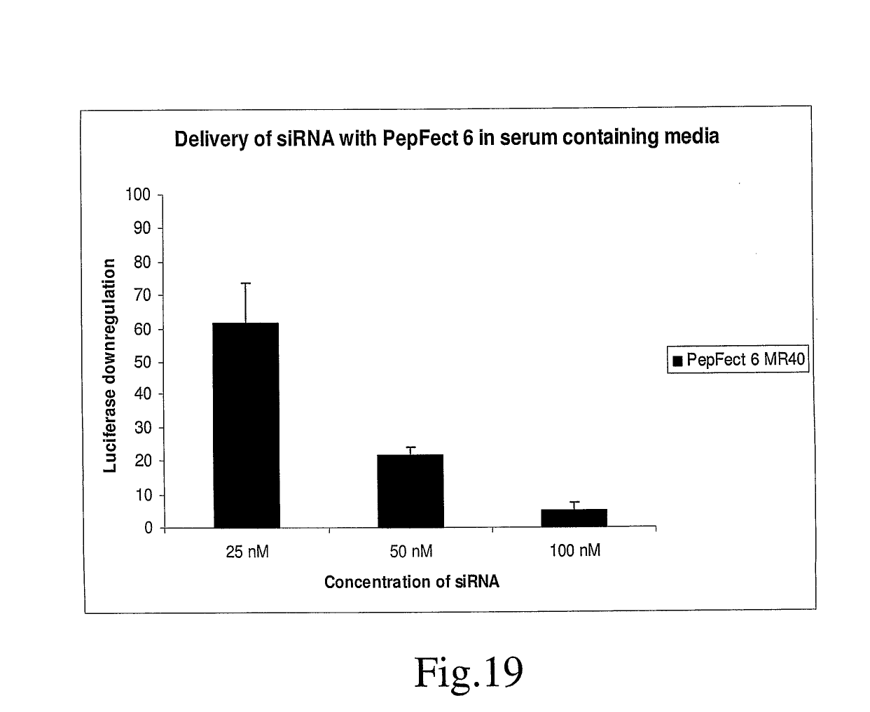

In this example, the CPPs are instead of, or in addition to, being modified with a stearic acid entity, also conjugated to a lysine tree bearing four chloroquine analogues (FIG. 5.) that facilitates release of the peptides from vesicular compartments. The constructs are not only active for transportation of ON compounds acting in the nucleus of cells, but can additionally be efficiently utilized for the delivery of cytoplasmically active ONs such as siRNAs (FIG. 16 and FIG. 17). In fact, both PepFect 5 and PepFect 6, and in particular the latter peptide, is significantly more active than Lipofectamine 2000 for the delivery of siRNAs in various cell types (FIG. 16-18). While Lipofectamine/siRNA complexes rarely generates more than 80% down-regulation of gene expression at any given siRNA concentration, both PepFects complexed with siRNA confers almost a complete RNAi at low siRNA concentrations. In addition, Pepfect 6 is efficient also in full growth, serum containing media (FIG. 19). The reason for this might be that it is double modified with both a stearic acid moiety and a fluoroquine tree compared to PepFect 5 that lacks the stearyl moiety (FIG. 5).

### Example 8

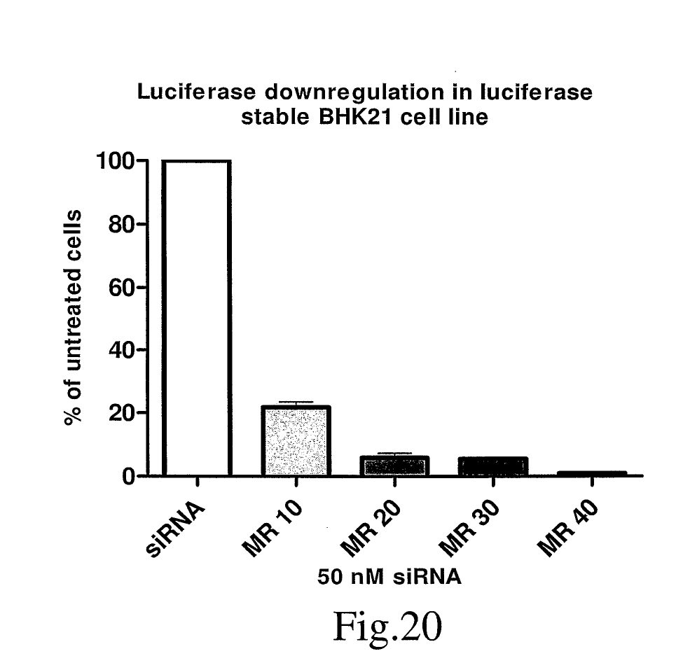

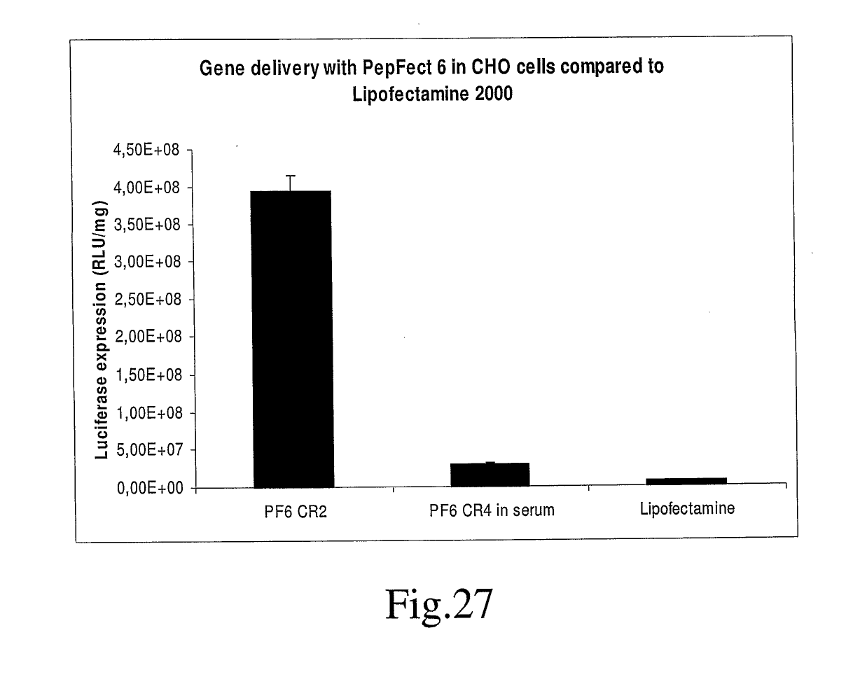

### (FIG. 20-28) PepFect Transfections: Homogenous, Stable and Works in Difficult Cells

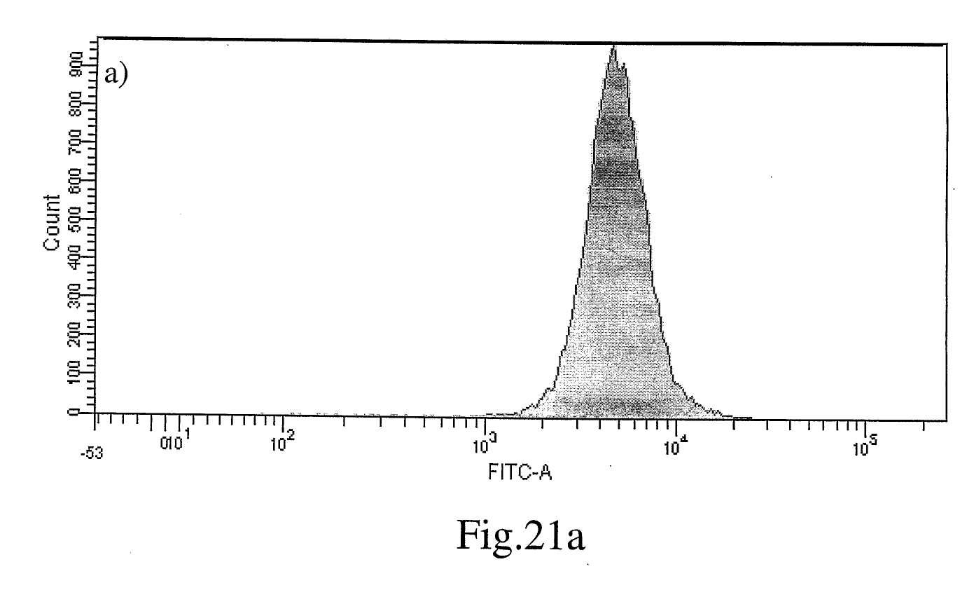

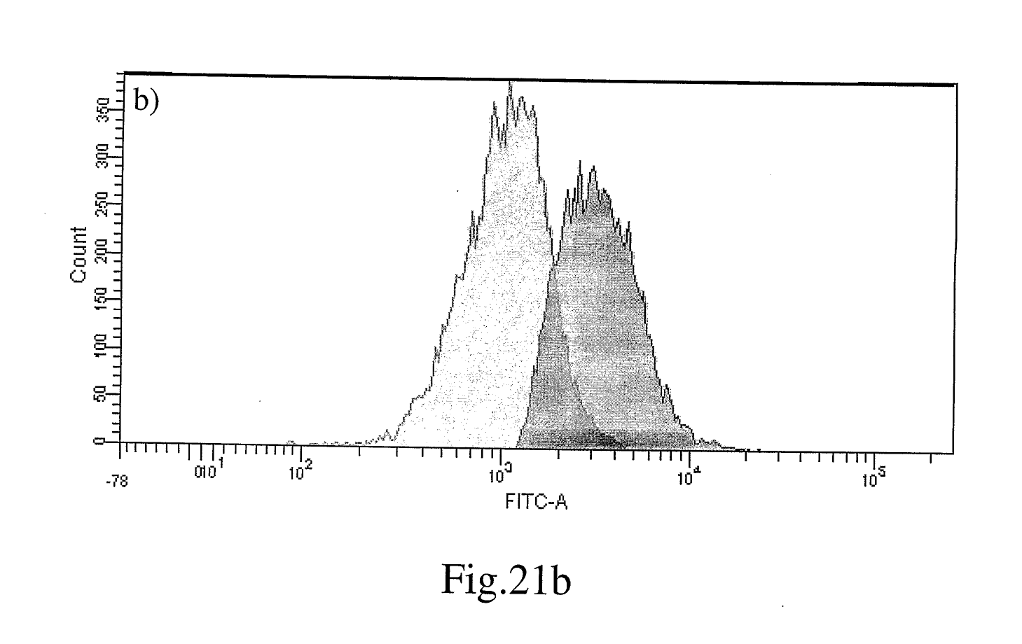

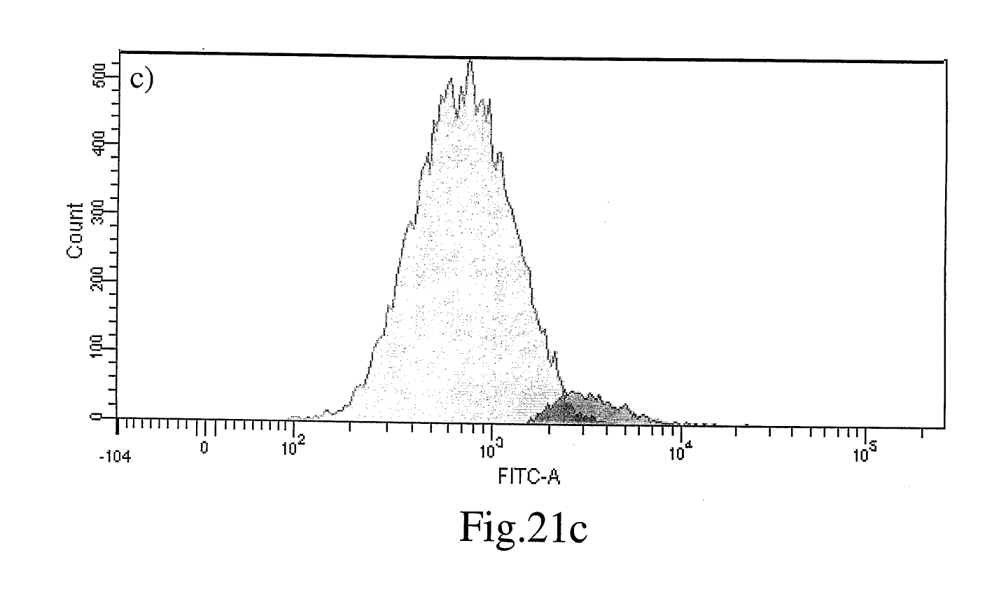

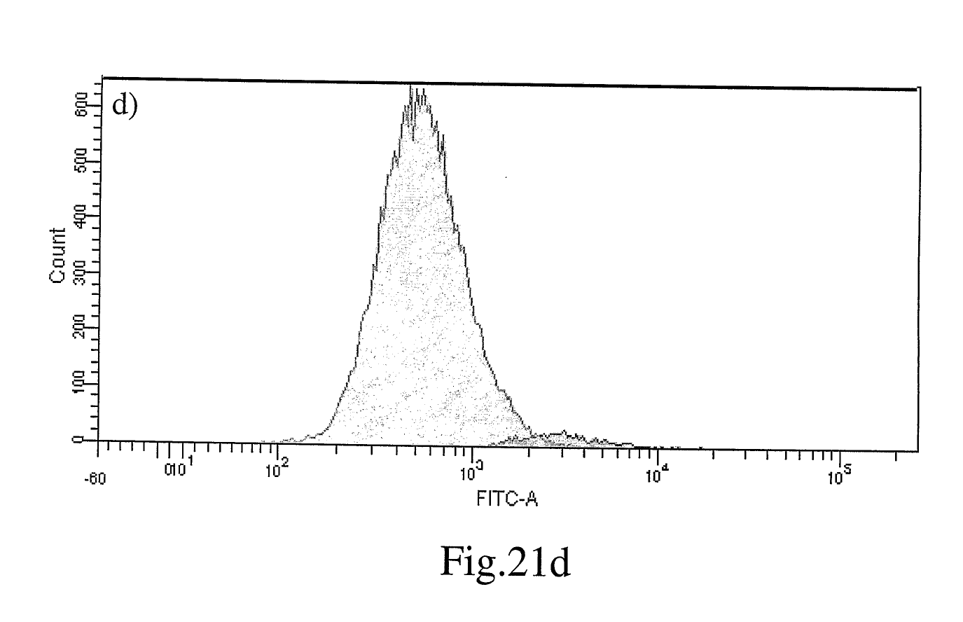

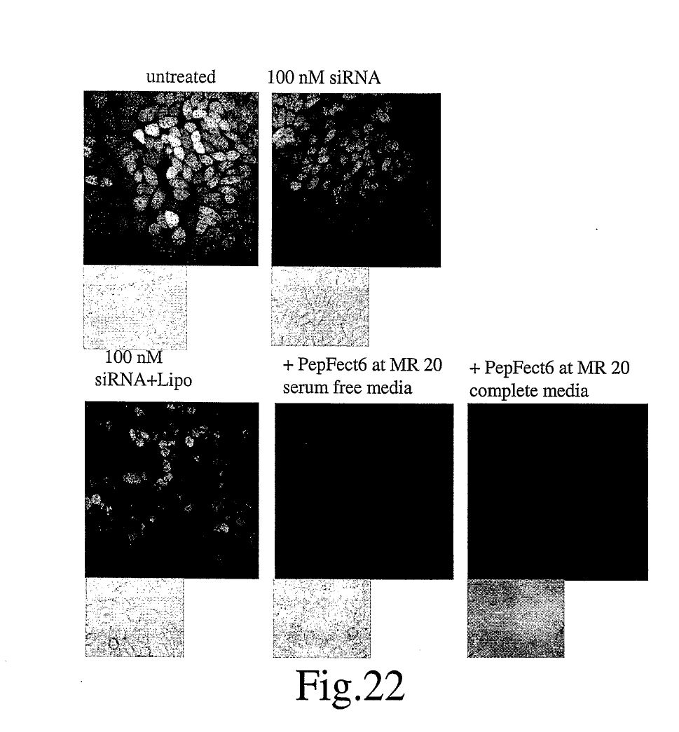

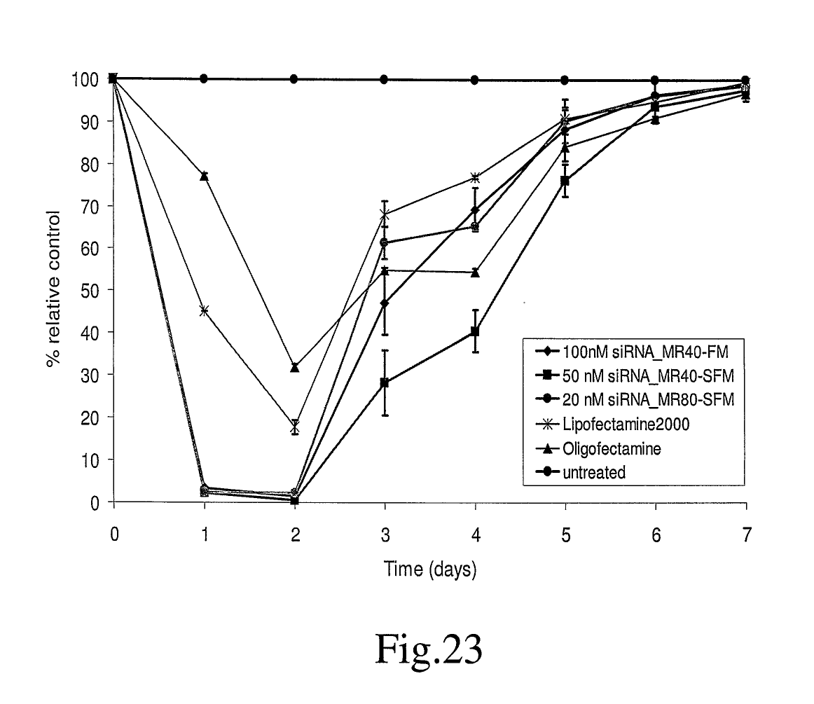

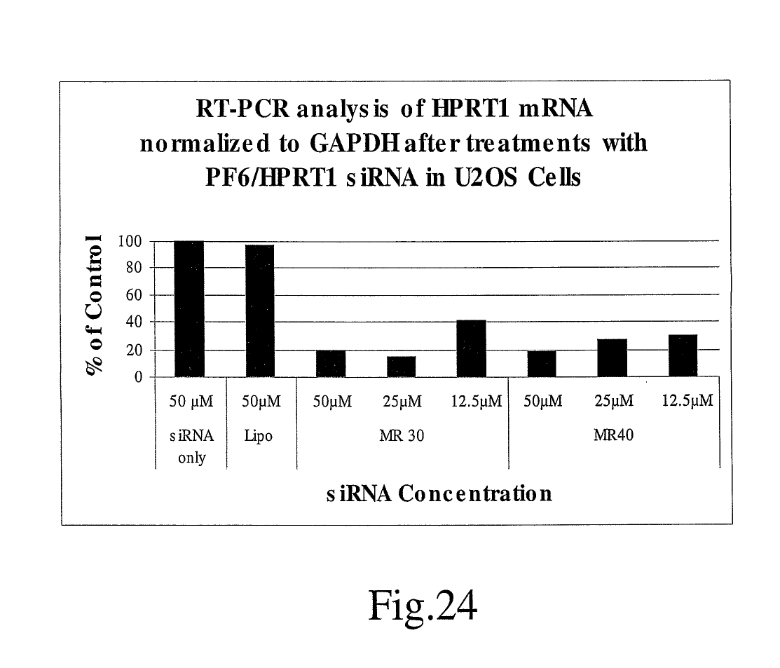

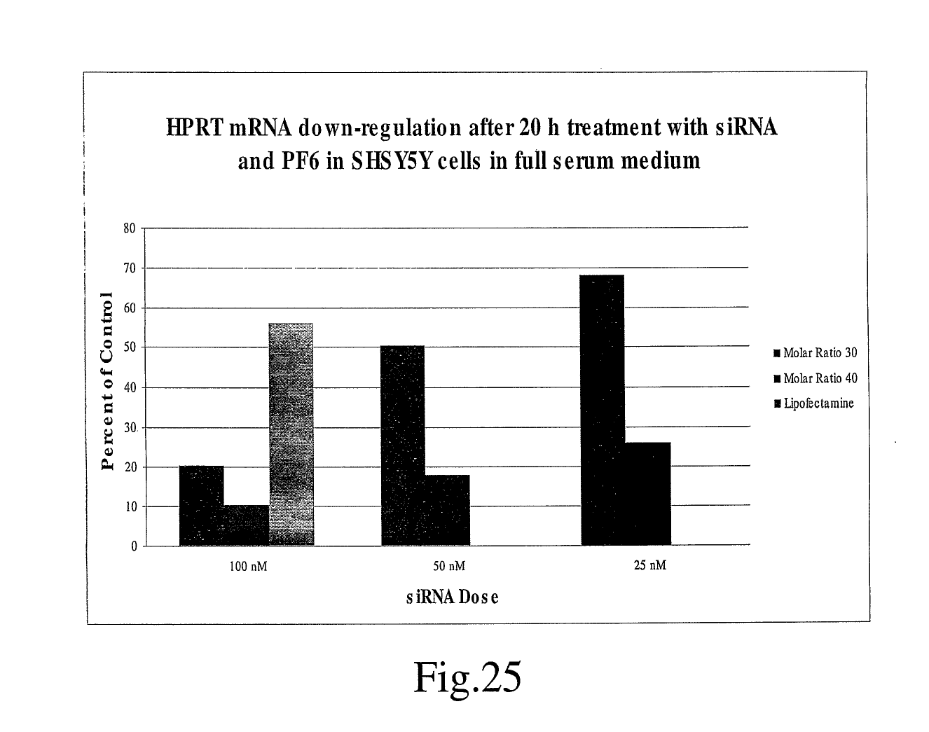

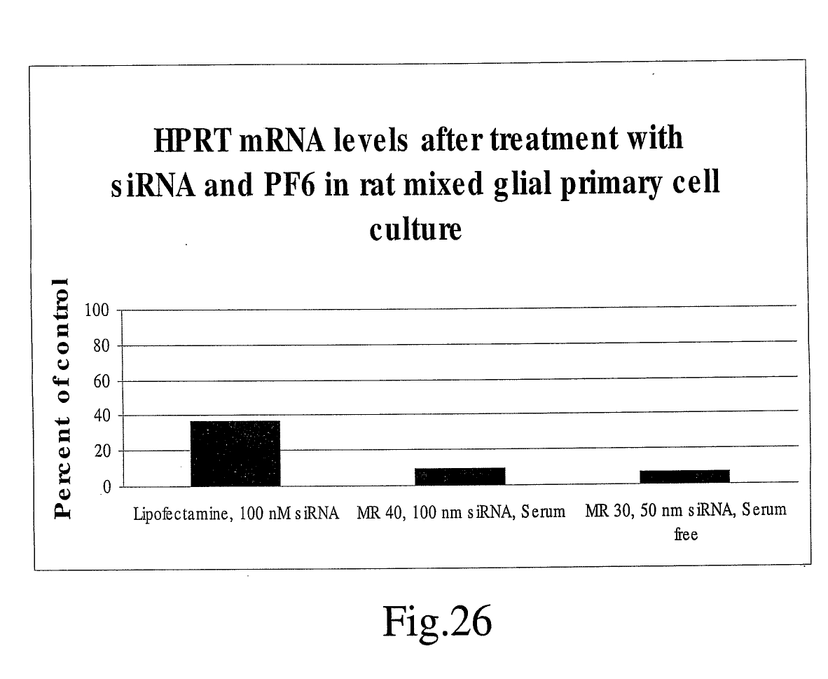

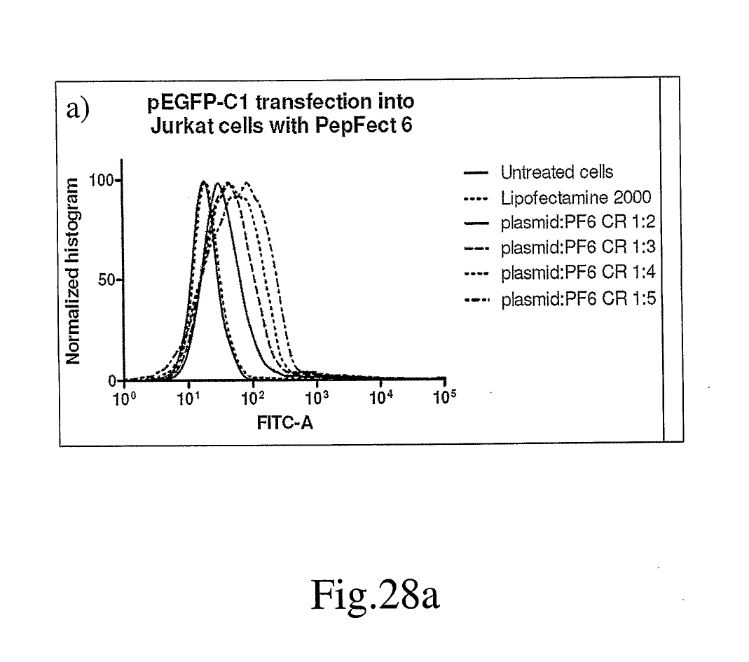

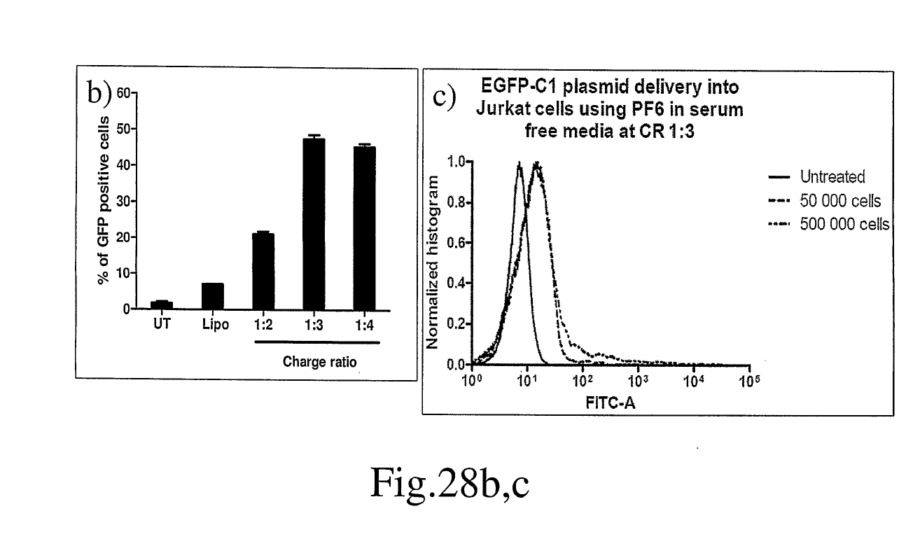

A user friendly reagent should tolerate minor variations in for instance molar ratio in complex formation. PF 6 works almost equally well at different molar ratio (FIG. 20): RNAi in BHK21 cells after 24 h treatment with PF6 complexed with 50 nM siRNA. Even at such low MR as 10, it is possible to obtain more than 80% down-regulation of luciferase in luciferase-stable BHK21 cells. This is a stronger RNAi effect than what is typically possible to obtain with Lipofectamine 2000 or any other Liposome-based delivery system. Interestingly, between MR20 and MR40, the difference in response is rather low. This makes the delivery system more user-friendly compared to Lipofectamine 2000, where small changes in amounts taken for transfections have drastic impact on the transfection efficiency. In addition to the high efficiency in serum containing media (FIG. 21-27), the PepFect 6 construct transfect entire cell populations and not only the dividing cells (FIG. 21). This is further visualised by fluorescence microscopy in FIG. 22. Moreover, longer siRNA, so called dicer-substrate, can also be efficiently delivered by PF6 (FIG. 24). FIG. 23 shows analysis of RNAi decay kinetics following a single siRNA treatment in EGFP-CHO cells. PF6/siRNA particles were formulated at the given concentrations of siRNA and treatment where performed in serum or serum free media. The effect was then compared to the RNAi induced by 100 nM siRNA complexed with Lipofectamine 2000 or Oligofectamine. The results clearly show that independent on siRNA concentration, PF6 induces almost complete RNAi already after 24 h. This should be compared to a 20% and 55% knock-down observed with Oligofectamine and Lipofectamine 2000, respectively. Furthermore, at optimal conditions, the RNAi response persists for 4-5 days when using PF6. Finally, PepFect6 is able to transfect a number of very “difficult to transfect” cells including SHSY-5Y (FIG. 25), N2a, rat primary glia cells (FIG. 26) and Jurkat suspension cells (FIG. 28). The above described properties, in combination with the lower toxicity compared to Lipofectamine 2000 or Oligofectamine, makes this particular vector highly unique.

### Example 9

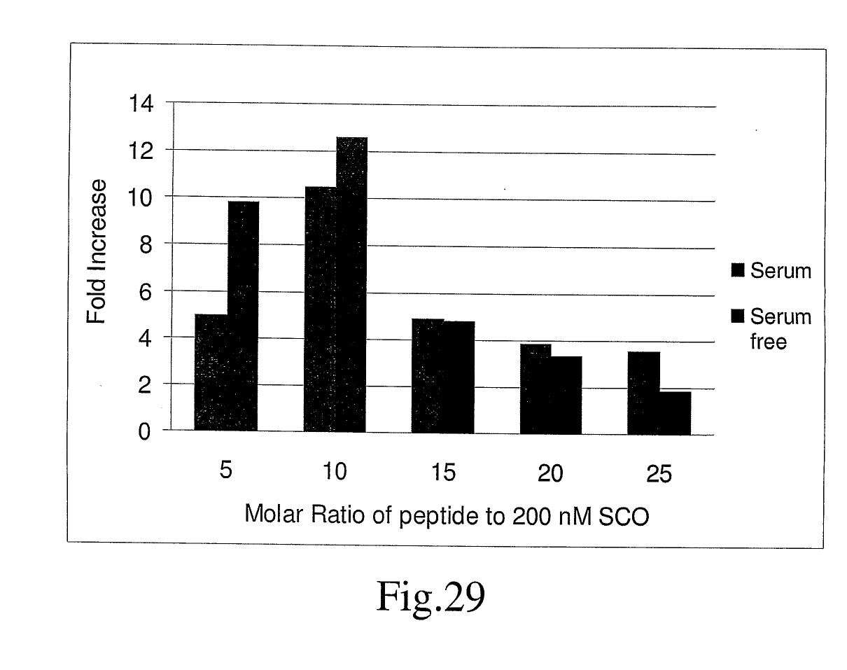

### (FIG. 29): Alternative Ring System Modification

In FIG. 29. we demonstrate that the entity A, does not have to be a quinoline analog. Naphthalene derivates and similar ring structures execute similar function. Splice correction in HeLa cells after treatment with a PF5 analogue complexed with SCOs. In this case, TP10 with lysine branching orthogonally with four copies of 1-naftoxy propanoic acid was used instead of the quinoline analogue, in order to assess the importance of the fusogenic properties. The results show that a 12-fold increases in splicing, compared to the 100-fold increase observed for PF5 previously.

### Example 10

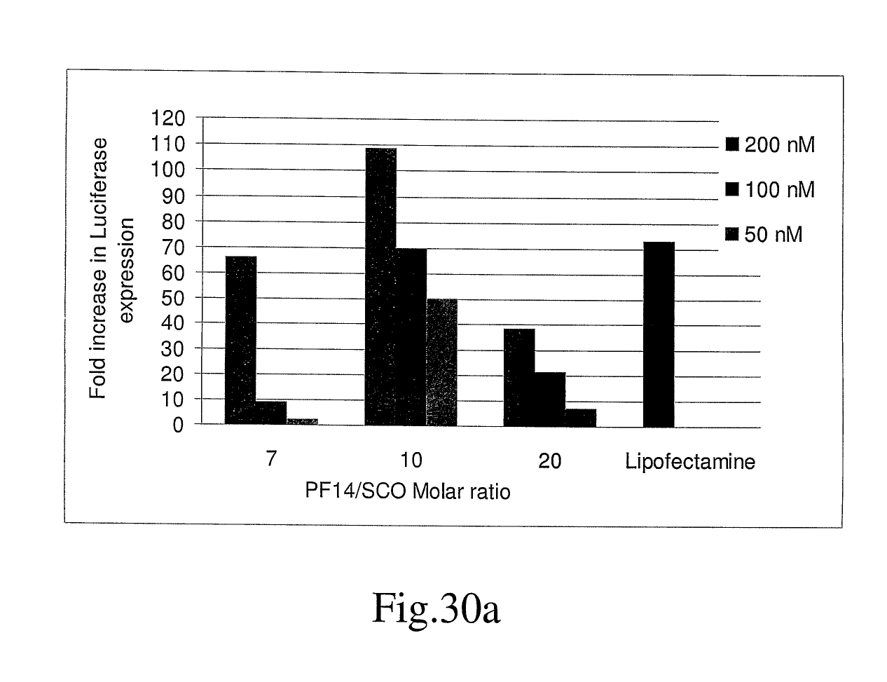

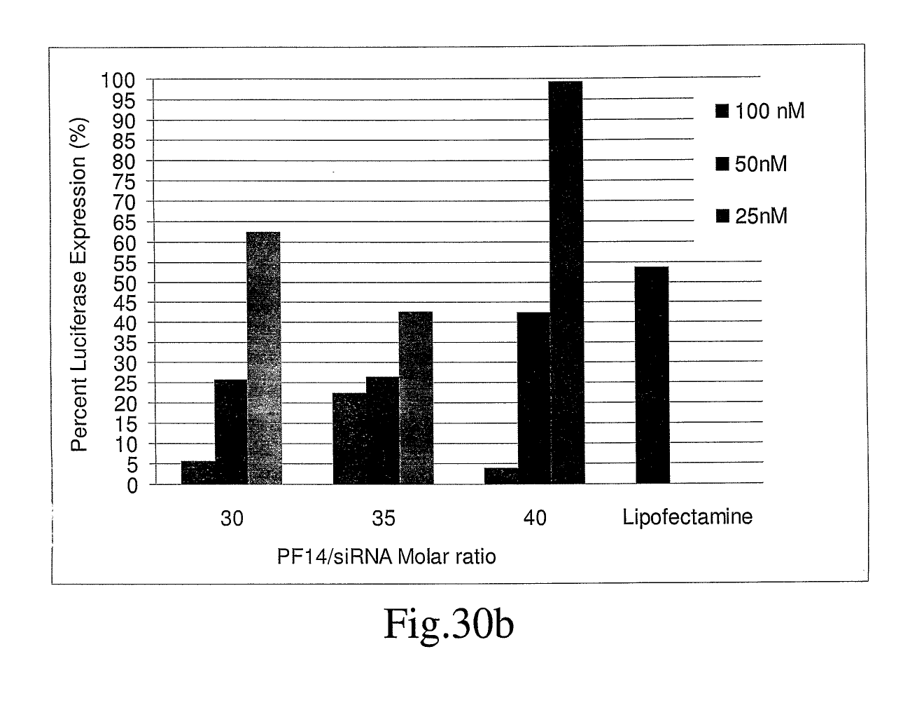

### (FIG. 30): Novel Cell-Penetrating Sequence: PepFect 14

Several different cell penetrating peptides have been tested in the PepFect delivery system and here is an example of a novel sequence with attachment of stearyl called Pepfect 14. PepFect 14 is able to effectively deliver both siRNA and SCO also in the presence of serum as FIG. 30 shows.

### Example 11

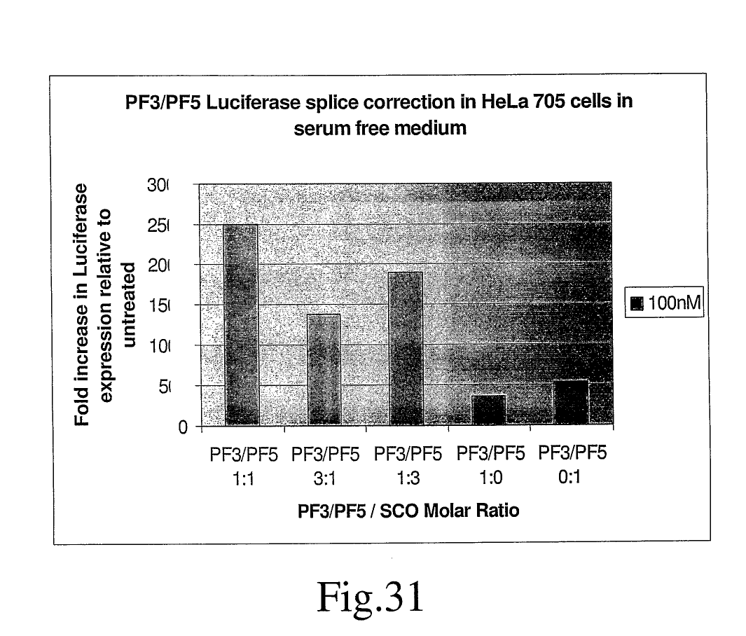

### (FIG. 31): PepFect Delivery Systems can be Mixed for Additive Effect

In addition, the PepFect delivery system can be added together as shown in FIG. 31, PF3 and PF5 in different ratio works synergistic and can deliver SCO better than the two PepFects by themselves. In addition, compositions of two or more PepFect delivery systems may similarly be mixed for additional properties such as targeting or prolonged half-life.

### Example 12

### Synthesis of Novel Amino-Chloroquine Derivative, N-(2-aminoethyl)-N-methyl-N′-[7-(trifluoromethyl)-quinolin-4-yl]ethane-1,2-diamine

A mixture of 3.8 g (16.3 mmol) 4-chloro-7-(trifluoromethyl)quinoline and 12 times molar excess of N-methyl-2,2′-diaminodiethylamine (25 ml) in a 50 mL round-bottom flask equipped with a magnetic stirrer is heated using PEG 400 bath from room temperature to 80° C. over 2.5 h with stirring, then temperature is raised to 130° C. over the period of 3 h, and finally heated 2.5 h at 140° C. The reaction mixture is cooled down to room temperature, and cold DCM is added, causing immediate precipitation, which is filtered off. The organic layer is washed twice with 5% aqueous NaHCO₃, then washed twice by water. The organic phase is dried over anhydrous MgSO₄, and solvent is removed under reduced pressure (rotavapor) and the residue is left in freeze-drier. Weight 4.5 g (MW 312.3) 14.4 mmol, 83% yield of crude product which is used for conjugation to peptides without further purification.

Coupling of N-(2-aminoethyl)-N-methyl-N′-[7-(trifluoromethyl)-quinolin-4-yl]ethane-1,2-diamine to the succininic acid modified side-chains of multiple lysine residues, providing multiple copies of QN analogue covalently bound to molecule of carrier. Activation of solid supported free carboxyl groups is achieved with 3 equivalents of TBTU/HOBt and 6 equivalents of DIEA. An excess (2-5 equivalents) of novel derivative of chloroquine to be attached, N-(2-aminoethyl)-N-methyl-N′-[7-(trifluoromethyl)-quinolin-4-yl]ethane-1,2-diamine, is dissolved in DMF and added to the peptide-resin simultaneously with activating reagent, to couple QN analogue via its free amino group group to the activated resin. To assure complete coupling, this reaction is allowed to run for prolonged period of time (typically overnight) as it can not be monitored by Kaiser test.

### REFERENCES

1. Cross, D. & Burmester, J. K. Gene therapy for cancer treatment: past, present and future. Clin Med Res 4, 218-27 (2006).
2. Kim, D. H. & Rossi, J. J. Strategies for silencing human disease using RNA interference. Nat Rev Genet 8, 173-84 (2007).
3. Mercatante, D. R., Sazani, P. & Kole, R. Modification of alternative splicing by antisense oligonucleotides as a potential chemotherapy for cancer and other diseases. Curr Cancer Drug Targets 1, 211-30 (2001).
4. EL-Andaloussi, S., Holm, T. & Langel, Ü. Cell-penetrating peptides: mechanisms and applications. Curr Pharm Des 11, 3597-611 (2005).
5. Mae, M. & Langel, U. Cell-penetrating peptides as vectors for peptide, protein and oligonucleotide delivery. Curr Opin Pharmacol 6, 509-14 (2006).
6. El-Andaloussi, S., Jarver, P., Johansson, H. J. & Langel, U. Cargo-dependent cytotoxicity and delivery efficacy of cell-penetrating peptides: a comparative study. Biochem J 407, 285-92 (2007).
7. Abes, S. et al. Vectorization of morpholino oligomers by the (R-Ahx-R)4 peptide allows efficient splicing correction in the absence of endosomolytic agents. J Control Release 116, 304-13 (2006).
8. Bendifallah, N. et al. Evaluation of cell-penetrating peptides (CPPs) as vehicles for intracellular delivery of antisense peptide nucleic acid (PNA). Bioconjug Chem 17, 750-8 (2006).
9. Lundberg, P., El-Andaloussi, S., Sutlu, T., Johansson, H. & Langel, U. Delivery of short interfering RNA using endosomolytic cell-penetrating peptides. Faseb J 21, 2664-71 (2007).
10. Derossi, D., Joliot, A. H., Chassaing, G. & Prochiantz, A. The third helix of the Antennapedia homeodomain translocates through biological membranes. J Biol Chem 269, 10444-50 (1994).
11. Soomets, U. et al. Deletion analogues of transportan. Biochim Biophys Acta 1467, 165-76 (2000).
12. El-Andaloussi, S., Johansson, H. J., Holm, T. & Langel, U. A Novel Cell-penetrating Peptide, M918, for Efficient Delivery of Proteins and Peptide Nucleic Acids. Mol Ther 15, 1820-6 (2007).
13. Wender, P. A. et al. The design, synthesis, and evaluation of molecules that enable or enhance cellular uptake: peptoid molecular transporters. Proc Natl Acad Sci USA 97, 13003-8 (2000).
14. Kang, S. H., Cho, M. J. & Kole, R. Up-regulation of luciferase gene expression with antisense oligonucleotides: implications and applications in functional assay development. Biochemistry 37, 6235-9 (1998).
15. Morris, M. C., Vidal, P., Chaloin, L., Heitz, F. & Divita, G. A new peptide vector for efficient delivery of oligonucleotides into mammalian cells. Nucleic Acids Res 25, 2730-6 (1997).
16. Morris, M. C., Chaloin, L., Mery, J., Heitz, F. & Divita, G. A novel potent strategy for gene delivery using a single peptide vector as a carrier. Nucleic Acids Res 27, 3510-7 (1999).
17. Du, L., Pollard, J. M. & Gatti, R. A. Correction of prototypic ATM splicing mutations and aberrant ATM function with antisense morpholino oligonucleotides. Proc Natl Acad Sci USA 104, 6007-12 (2007).
18. Garcia-Blanco, M. A., Baraniak, A. P. & Lasda, E. L. Alternative splicing in disease and therapy. Nat Biotechnol 22, 535-46 (2004).19. Si M-L, Zhu S, Wu H, Lu Z, Wu F, Mo Y Y. miR-21-mediated tumor growth. Oncogene 26: 2799-2803. (2007).

1. A system for intracellular cargo delivery comprising:
(a) at least one component A selected from the group consisting of:
(i) aliphatic linear or branched moieties with at least 4 carbon atoms, and
(ii) cyclic ring systems comprising 2-4 rings, which may contain several hetero atoms selected from the group consisting of N, S, O and P; and
(b) a cell penetrating peptide B and/or a non-peptide analog thereof; wherein said at least one component A is attached to said peptide B and/or non-peptide analog thereof.

2. The system according to claim 1 wherein said at least one component A and said peptide B have at least one functional group each for the attachment.

3. The system according to claim 1, further comprising at least one component C, which is a targeting moiety.

4. The system according to claim 1, further comprising a cargo.

5. The system according to claim 1, wherein said at least one component A, one or more components C and one or more cargos are attached to a side-chain, and/or at the N-terminal and/or at the C-terminal of said peptide B and/or or a non-peptide analogue thereof.

6. The system according to claim 1, comprising more than one peptide B.

7. The delivery system according to claim 1, wherein the entity A is selected from the group consisting of a hydrophobic entity and a 2-4 ring system which comprises a heteroatom and/or is substituted.

8. The system according to claim 1, wherein at least one of the components A, C and the cargo are attached with a spacer arm.

9. The system according to claim 1, wherein the peptide B comprises one or more peptides selected from the group consisting of the following sequences:

| | |
|---|---|
| SEQ ID NO: 1 | AGYLLGKINLKALAALAKKIL; |
| SEQ ID NO: 2 | AGYLLGKLLOOLAAAALOOLL; |
| SEQ ID NO: 3 | RKKRKKKRXRHXRHXRHXR; |
| SEQ ID NO: 4 | MVTVLFRRLRIRRACGPPRVRV; |
| SEQ ID NO: 5 | RKKRKKK(HXH)4; |
| SEQ ID NO: 6 | LLOOLAAAALOOLL; |
| SEQ ID NO: 7 | RQIKIWFQNRRMKWKK |
| SEQ ID NO: 8 | RRRRRRRRR; |
| SEQ ID NO: 9 | MVTVLFRRLRIRRACGPPRVRV; |
| SEQ ID NO: 10 | GALFLGFLGAAGSTMGAWSQPKKKRKV; |
| SEQ ID NO: 11 | FILFILFILGGKHKHKHKHKHK; |
| SEQ ID NO: 12 | FILFILFILGKGKHKHKHKHKHK; |
| SEQ ID NO: 13 | FILFILFILGKGKHRHKHRHKHR; |
| SEQ ID NO: 14 | AGYLLGKINLKALAALAKKIL; |
| SEQ ID NO: 15 | GDAPFLDRLRRDQKSLRGRGSTL; |
| SEQ ID NO: 16 | PFLDRLRRDQKSLRGRGSTL; |
| SEQ ID NO: 17 | PFLNRLRRDQKSLRGRGSTL; |
| SEQ ID NO: 18 | PFLDRLRRNQKSLRGRGSTL; |
| SEQ ID NO: 19 | PFLNRLRRNQKSLRGRGSTL; |
| SEQ ID NO: 20 | PFLNRLRRNLKSLRGRLSTL; |
| SEQ ID NO: 21 | PFLDRKRRDQKSLRGRGSTL; |
| SEQ ID NO: 22 | RHRHRHHHGGPFLDRLRRDQKSLRGRGSTL; |
| SEQ ID NO: 23 | PNNVRRDLDNLHACLNKAKLTVSRMVTSLLEK; |
| SEQ ID NO: 24 | PNNVRRDLDNLHAMLNKAKLTVSRMVTSLLEK; |
| SEQ ID NO: 25 | PNNVRRDLNNLHAMLNKAKLTVSRMVTSLLQK; |
| SEQ ID NO: 26 | PFLNRLRRNLKSLRGRLSTL; |
| SEQ ID NO: 27 | PFLNRKRRNLKSLRGRLSTL; |
| and | |
| SEQ ID NO: 28 | INLKALAALAKKIL. |

10. The system according to claim 1, wherein the peptide B is a peptide that contains a sequence of the formula Ny₁-Bx₁-Ny₂-Bx₂-Ny₃, where B is a basic amino acid and N is a neutral amino acid and x₁, x₂, y₁, y₂ and y₃ are integers between 2 and 8.

11. The system according to claim 1, wherein the peptide B is selected from the group consisting of LLOOLAAAALOOLL [SEQ ID No 6], AGYLLGKLLOOLAAAALOOLL [SEQ ID No 2], INLKALAALAKKIL [SEQ ID No 28], AGYLLGKINLKALAALAKKIL [SEQ ID No 1] and deletions, additions insertions and substitutions of amino acids thereof.

12. The system according to claim 1, wherein the C-terminus of the cell penetrating peptide B and/or the non-peptide analogue thereof is modified.

13. The system according to claim 1, wherein the cargo is selected from the group consisting of oligonucleotides, modified versions of oligonucleotides plasmids, variations of plasmids, and synthetic nucleotide analogues.

14. The system according to claim 1, wherein the cargo is linked to one or more members selected from the group consisting of a fluorescent marker, a cell- or tumor-homing peptide, an aptamer, a receptor ligand, a spacer comprising a cleavable site coupled to an inactivating peptide, peptide ligands, cytotoxic peptides, bioactive peptides, diagnostic agents, proteins, and pharmaceuticals.

15. The system according to claim 1, further comprising at least one imaging agent and/or labelling molecule.

16. The system according to claim 15, wherein the at least one labelling molecule is a molecular beacon selected from the group consisting of quenched fluorescence based beacons and FRET technology based beacons, for labelling or quantification of intracellular mRNA.

17. The system according to claim 1 further comprising a circulation clearance modifiers.

18. A method of diagnosing a disease in a subject, comprising administering said system according to claim 1 to a cell of said subject.

19. A composition comprising more than one delivery system according to claim 1, wherein the delivery systems comprise different components A, and/or different penetrating peptides B and/or a non-peptide analogs thereof, and/or different targeting moieties C and/or different cargos, and optionally a circulation clearance modifier.

20. The composition according to claim 19, wherein the delivery systems comprise at least two different penetrating peptides B and/or non-peptide analogs thereof and optionally a circulation clearance modifiers.

21. A pharmaceutical composition comprising one or more delivery systems according to claim 1, wherein the delivery systems comprise different components A, and/or different peptides B, and/or different targeting moieties C and/or different cargos and optionally a circulation clearance modifier.

22. A material covered with one or more of the delivery systems according to claim 1.

23. A material having one or more of the delivery systems according to claim 1 incorporated into the material.

24. A peptide that contains a sequence of the formula Ny₁-Bx₁-Ny₂-Bx₂-Ny₃, where B is a basic amino acid and N is a neutral amino acid and x₁, x₂, y₁, y₂ and y₃ are integers between 2 and 8.

25. The peptide of claim 24, wherein the entity B is selected from the group consisting of LLOOLAAAALOOLL [SEQ ID No 6], AGYLLGKLLOOLAAAALOOLL [SEQ ID No 2], INLKALAALAKKIL [SEQ ID No 28], AGYLLGKINLKALAALAKKIL [SEQ ID No 1] and deletions, additions, insertions and substitutions of amino acids thereof.

26. The delivery system according to claim 7, wherein said hydrophobic entity is a fatty acid with 10-30 carbon atoms or a derivate thereof

27. The delivery system of claim 26, wherein said fatty acid is a stearic acid or a C18 derivate thereof.

28. The delivery system of claim 27, wherein said stearic acid or said C18 derivate thereof is selected from the group consisting of lauric-, myristic-, palmitic-, arachidic- and behenic acid.

29. The delivery system according to claim 7, wherein said 2-4 ring system is selected from the group consisting of quinoline and naphthalene analogues.

30. The system of claim 13, wherein said oligonucleotides or modified versions of oligonucleotides are single strand oligonucleotides or double-strand oligonucleotides.

31. The system of claim 30, wherein said single strand oligonucleotides are single strand oligonucleotides selected from the group consisting of DNA, RNA, PNA, LNA and synthetic oligonucleotides.

32. The system of claim 30, wherein said double-strand oligonucleotides are selected from the group consisting of siRNA, shRNA and decoy dsDNA.

33. The system of claim 14, wherein said pharmaceuticals are anticancer drugs or antibiotics.

34. The system according to claim 17, wherein said circulation clearance modifier is polyethylene glycol (PEG).

35. A method of targeting delivery of a cargo to a cell comprising administering the system of claim 1 to said cell.

36. The composition according to claim 19, wherein said circulation clearance modifier is polyethylene glycol (PEG).

37. The composition according to claim 20, wherein said circulation clearance modifier is polyethylene glycol (PEG).


SEQUENCE LISTING (28 sequences)

SEQ ID NO: 1 - protein, 21 aa, Artificial Sequence
  Synthetic peptide
AGYLLGKLLXXLAAAALXXLL

SEQ ID NO: 2 - protein, 21 aa, Artificial Sequence
  Synthetic peptide
AGYLLGKINLKALAALAKKIL

SEQ ID NO: 3 - protein, 19 aa, Artificial Sequence
  Synthetic peptide
RKKRKKKRXRHXRHXRHXR

SEQ ID NO: 4 - protein, 22 aa, Artificial Sequence
  Synthetic peptide
MVTVLFRRLRIRRACGPPRVRV

SEQ ID NO: 5 - protein, 19 aa, Artificial Sequence
  Synthetic peptide
RKKRKKKHXHHXHHXHHXH

SEQ ID NO: 6 - protein, 14 aa, Artificial Sequence
  Synthetic peptide
LLXXLAAAALXXLL

SEQ ID NO: 7 - protein, 16 aa, Artificial Sequence
  Synthetic peptide
RQIKIWFQNRRMKWKK

SEQ ID NO: 8 - protein, 9 aa, Artificial Sequence
  Synthetic peptide
RRRRRRRRR

SEQ ID NO: 9 - protein, 22 aa, Artificial Sequence
  Synthetic peptide
MVTVLFRRLRIRRACGPPRVRV

SEQ ID NO: 10 - protein, 27 aa, Artificial Sequence
  Synthetic peptide
GALFLGFLGAAGSTMGAWSQPKKKRKV

SEQ ID NO: 11 - protein, 22 aa, Artificial Sequence
  Synthetic peptide
FILFILFILGGKHKHKHKHKHK

SEQ ID NO: 12 - protein, 23 aa, Artificial Sequence
  Synthetic peptide
FILFILFILGKGKHKHKHKHKHK

SEQ ID NO: 13 - protein, 23 aa, Artificial Sequence
  Synthetic peptide
FILFILFILGKGKHRHKHRHKHR

SEQ ID NO: 14 - protein, 21 aa, Artificial Sequence
  Synthetic peptide
AGYLLGKINLKALAALAKKIL

SEQ ID NO: 15 - protein, 23 aa, Artificial Sequence
  Synthetic peptide
GDAPFLDRLRRDQKSLRGRGSTL

SEQ ID NO: 16 - protein, 20 aa, Artificial Sequence
  Synthetic peptide
PFLDRLRRDQKSLRGRGSTL

SEQ ID NO: 17 - protein, 20 aa, Artificial Sequence
  Synthetic peptide
PFLNRLRRDQKSLRGRGSTL

SEQ ID NO: 18 - protein, 20 aa, Artificial Sequence
  Synthetic peptide
PFLDRLRRNQKSLRGRGSTL

SEQ ID NO: 19 - protein, 20 aa, Artificial Sequence
  Synthetic peptide
PFLNRLRRNQKSLRGRGSTL

SEQ ID NO: 20 - protein, 20 aa, Artificial Sequence
  Synthetic peptide
PFLNRLRRNLKSLRGRLSTL

SEQ ID NO: 21 - protein, 20 aa, Artificial Sequence
  Synthetic peptide
PFLDRKRRDQKSLRGRGSTL

SEQ ID NO: 22 - protein, 30 aa, Artificial Sequence
  Synthetic peptide
RHRHRHHHGGPFLDRLRRDQKSLRGRGSTL

SEQ ID NO: 23 - protein, 32 aa, Artificial Sequence
  Synthetic peptide
PNNVRRDLDNLHACLNKAKLTVSRMVTSLLEK

SEQ ID NO: 24 - protein, 32 aa, Artificial Sequence
  Synthetic peptide
PNNVRRDLDNLHAMLNKAKLTVSRMVTSLLEK

SEQ ID NO: 25 - protein, 32 aa, Artificial Sequence
  Synthetic peptide
PNNVRRDLNNLHAMLNKAKLTVSRMVTSLLQK

SEQ ID NO: 26 - protein, 20 aa, Artificial Sequence
  Synthetic peptide
PFLNRLRRNLKSLRGRLSTL

SEQ ID NO: 27 - protein, 20 aa, Artificial Sequence
  Synthetic peptide
PFLNRKRRNLKSLRGRLSTL

SEQ ID NO: 28 - protein, 14 aa, Artificial Sequence
  Synthetic peptide
INLKALAALAKKIL